(19) United States
(12) Patent Application Publication
(10) Pub. No.: US 2026/0108730 A1
(43) Pub. Date: Apr. 23, 2026

(54) METHODS FOR MODULATING TEMPORAL INFRASTRUCTURE OF PULSED ELECTRIC FIELDS

(71) Applicant: Virginia Tech Intellectual Properties, Inc., Blacksburg, VA (US)

(72) Inventors: Kenneth N. Aycock, Blacksburg, VA (US); Rafael V. Davalos, Aptos, CA (US)

(21) Appl. No.: 19/368,292

(22) Filed: Oct. 24, 2025

Related U.S. Application Data
(63) Continuation of application No. 17/535,742, filed on Nov. 26, 2021, now Pat. No. 12,485,279.
(60) Provisional application No. 63/118,098, filed on Nov. 25, 2020.

(51) Int. Cl. A61N 1/32; A61B 18/00; A61N 1/04

(52) U.S. Cl. CPC A61N 1/327; A61N 1/0416; A61B 2018/00577; A61B 2018/00613; A61B 2018/00761

(57) ABSTRACT

High-frequency irreversible electroporation (H-FIRE) is a tissue ablation modality employing bursts of electrical pulses in a positive phase-interphase delay-negative phase-interpulse delay pattern. Despite accumulating evidence suggesting the significance of these delays, their effects on therapeutic outcomes from clinically-relevant H-FIRE waveforms have not been studied extensively. The present invention provides methods of pulse delivery, including delays, that mitigate bubble formation and/or minimize the risk of arcing, such as due to the presence of bubbles, and/or minimize muscle stimulation are described herein.

### CROSS-REFERENCE TO RELATED APPLICATIONS

This application is a Continuation of U.S. patent application Ser. No. 17/535,742 filed Nov. 26, 2021, which application relies on the disclosure of and claims priority to and the benefit of the filing date of U.S. Provisional Application No. 63/118,098 filed on Nov. 25, 2020, which applications are hereby incorporated by reference herein in their entireties.

Additionally, the present application is related to U.S. Pat. Nos. 8,465,484, 8,814,860, 8,926,606, 8,992,517, 9,198,733, 9,283,051, 9,598,691, 9,867,652, 10,117,707, 10,154,874, 10,238,447, 10,245,098, 10,245,105, 10,272,178, 10,286,108, 10,292,755, 10,448,989, 10,470,822, 10,471,254, 10,537,379, 10,694,972, 10,702,326, 10,828,085, 10,828,086, 10,959,772; U.S. Patent Publication Nos. 2015/0289923, 2019/0029749, 2020/0093541, 2019/0175248, 2019/0223938, 2019/0232048, 2019/0233809, 2019/0256839, 2019/0282294, 2019/0328445, 2019/0351224, 2019/0376055, 2020/0046432, 2020/0093541, 2020/0197073, 2020/0260987, 2020/0323576, 2020/0405373, 2021/0023362, 2021/0052882, 2021/0022795, and 2021/0186600; International Patent Publication Nos. WO2009/134876, WO2010/118387, WO2010/151277, WO2011/047387, WO2012/0088149, WO2012/071526, WO2015/175570, and WO2020/061192; U.S. patent application Ser. Nos. 13/958,152, 17/277,662, and 17/338,960, each of which is incorporated by reference herein in its entirety.

### GOVERNMENT SUPPORT CLAUSE

This invention was made with government support under Grant No. P01CA207206 awarded by the National Institutes of Health (NIH) National Cancer Institute (NCI). The government has certain rights in the invention.

### BACKGROUND OF THE INVENTION

### Field of the Invention

The present invention relates to the field of electroporation treatment. More specifically, embodiments of the present invention relate to using pulsed electric fields to electroporate tissue, e.g., tumors, while minimizing and/or preventing muscle stimulation, non-targeted mechanical destruction of tissue, bubble formation, and/or electrical arcing.

### Description of Related Art

Electroporation is a biological phenomenon in which cells exhibit increased membrane permeability upon exposure to high amplitude electric fields. Increased permeabilization is presumably due to creation of defects in the cell membrane that increase transport of ionic species and macromolecules (Yarmush, M. L. et al., “Electroporation-Based Technologies for Medicine: Principles, Applications, and Challenges,” Annu. Rev. Biomed. Eng., vol. 16, no. 1, 295-320, 2014.). Electroporation can be implemented reversibly, whereby affected cells regain membrane integrity and recover following stimulus removal, or irreversibly, where cells die following treatment. Unlike conventional ablative therapies in which temperature is manipulated to nonspecifically denature proteins, IRE directly affects cellular membranes without significant local heating, leaving the underlying tissue architecture intact. Nonetheless, management of patients receiving IRE can be difficult. The long (70-100 μs) pulses of conventional IRE easily stimulate cardiac myocytes, pain receptors, and skeletal muscle fibers, resulting in muscle contractions and potential arrhythmias.

To further extend clinical capabilities of IRE, an alternative pulse delivery scheme termed high-frequency IRE (H-FIRE) exists (Arena, C. B. et al., “Theoretical Considerations of Tissue Electroporation With High-Frequency Bipolar Pulses,” IEEE Trans. Biomed. Eng., vol. 58, no. 5, 1474-1482, 2011.H-FIRE replaces the long monopolar pulses with bursts of short (1-10 μs) bipolar pulses following a positive phase-interphase delay (d₁)-negative phase-interpulse delay (d₂) pattern (FIGS. 1A-C) (Arena, 2011, IEEE Trans. Biomed. Eng.; Arena, C. B. et al., “High-frequency irreversible electroporation (H-FIRE) for non-thermal ablation without muscle contraction,” Biomed. Eng. Online, vol. 10, no. 102, 1-20, 2011.). By changing the method of pulse delivery in this way, nerve excitation and muscle contractions are substantially reduced (Arena, 2011, Biomed. Eng. Online; Partridge, B. R. et al., “High-Frequency Irreversible Electroporation for treatment of Primary Liver Cancer: A Proof-of-Principle Study in Canine Hepatocellular Carcinoma,” J. Vasc. Interv. Radiol., vol. 31, no. 3, 482-491.e4, March 2020. This is due to the local repolarization that occurs with each negative phase prior to activation of the minimum number of voltage-gated sodium channels (VGSCs) required for excitation.

Additionally, the short bipolar pulses constitute a shift towards higher frequencies, which translates to more predictable ablation geometries and reduced likelihood of electrical arcing (Bhonsle, S. P. et al., “Mitigation of impedance changes due to electroporation therapy using bursts of high-frequency bipolar pulses,” Biomed. Eng. (NY)., vol. 14, no. Suppl 3, 1-14, 2015.

A challenge that arises with H-FIRE waveforms is that lethal electric field thresholds (EFTs) are typically 1.5-3× higher than with IRE and are heavily dependent on the width of constitutive pulses and as such higher voltages are typically applied across the electrodes to achieve similar ablation volumes, which can have consequences on the amount of heat produced. A number of studies have explored non-pharmacological methods of lowering lethal EFTs for H-FIRE waveforms by modifying the delivery strategy or constitutive pulse width of burst waveforms. Sano et al. showed that with a fixed width of 2 μs, single bipolar pulses repeated at 25-100 Hz may exhibit lower EFTs than traditional bursts (Sano, M. B. et al., “Burst and continuous high frequency irreversible electroporation protocols evaluated in a 3D tumor model,” Phys. Med. Biol., vol. 63, no. 13, 2018.). Additionally, reducing the width of alternate polarity pulses lowered the EFT to roughly half the value of corresponding symmetric bursts (Sano, 2018), but the charge imbalance caused muscle contractions similar to those resulting from IRE (Sano, M. B. et al., “Reduction of Muscle Contractions During Irreversible Electroporation Therapy Using High-Frequency Bursts of Alternating Polarity Pulses: A Laboratory Investigation in an Ex Vivo Swine Model,” J. Vasc. Interv. Radiol., vol. 29, no. 6, 893-898.e4, June 2018.).

Investigations have shown that cell permeability and survival are also closely linked to the delays within (interphase delay, d₁) and between (interpulse delay, d₂) bipolar pulses (FIG. 1B) (Polajžer, T. et al., “Cancellation effect is present in high-frequency reversible and irreversible electroporation,” Bioelectrochemistry, vol. 132, 2020; Vižintin, A. et al., “Effect of interphase and interpulse delay in high-frequency irreversible electroporation pulses on cell survival, membrane permeabilization and electrode material release,” Bioelectrochemistry, vol. 134, August 2020.). By increasing interphase and interpulse delays, the time the membrane is exposed to a critical transmembrane potential (usually ˜1 V) is increased (Arena, 2011, Biomed. Eng. Online). Further, a complex relationship exists between these delays and electroporation effects that cannot be fully explained by assisted discharge, and that extensions of the interpulse delay alone may enhance biophysical outcomes from treatment (Vižintin, 2020). From a stimulation perspective, many studies have investigated methods to maximize efficiency of charge-balanced waveforms for functional stimulation purposes, offering insight into the effects of interphase gaps, multiple pulses in series, etc., but most of these investigations have been for much longer pulses than those of interest here (Butikofer, R. et al., “Electrocutaneous Nerve Stimulation-I: Model and Experiment,” IEEE Trans. Biomed. Eng., vol. BME-25, no. 6, 526-531, 1978; Butikofer, R. et al., “Electrocutaneous Nerve Stimulation-II: Stimulus Waveform Selection,” IEEE Trans. Biomed. Eng., vol. BME-26, no. 2, 69-75, 1979; Macherey, O. et al., “Asymmetric pulses in cochlear implants: Effects of pulse shape, polarity, and rate,” JARO-J. Assoc. Res. Otolaryngol., vol. 7, no. 3, 253-266, 2006.).

When an electric potential is established across an electrolytic medium such as tissue, current flows through the medium due to movement of ionic species (rather than electrons). Electrochemical events occurring at the electrode-tissue interface mediate the charge exchange between the metallic electrodes and electrolyte. Conventional irreversible electroporation (IRE) and high-frequency IRE (H-FIRE) are typically delivered using one or more pairs of monopolar or bipolar needle electrodes, which are made of stainless steel. Electrochemical interactions at the cathode include the reduction of water into hydrogen gas (bubbles) and hydroxide ions (basic pH). At the anode, the opposite occurs, resulting in production of oxygen gas (bubbles) and protons (acidic pH). Other electrochemical products are also formed at either electrode, but gas production due to electrolysis at either electrode can reduce clinical efficacy of the procedure.

First, electrolytic gas formation from ablation systems can cause microemboli that have been linked to ischemic cerebral events (Groen, M. H. A. et al., “In Vivo Analysis of the Origin and Characteristics of Gaseous Microemboli during Catheter-Mediated Irreversible Electroporation,” Europace, 2021, 23(1), 139-146.). These bubbles also impair the ability to visualize ablation formation on real-time clinical monitoring systems, particularly ultrasound. Finally, bubbles that are formed and remain adjacent to the electrode surface can break down upon further exposure to electric fields, creating an electrical arc. Electrical arcing is highly uncontrolled and can lead to very high stray currents and mechanical (physical) degradation of tissue, as well as other complications such as generator shutdown (treatment termination) or damage and heightened potential for induced cardiac arrhythmias.

For a given water molecule, twice the amount of hydrogen gas is formed relative to oxygen gas. Oxygen gas is also over twenty-six times more soluble in water as hydrogen gas, so O₂ gas may quickly dissolve. Thus, gaseous effects at the cathode tend to present more significant concerns relative to those at the anode. For conventional IRE and monophasic bursts using a single electrode pair, the cathode remains the same throughout pulse administration, e.g., throughout delivery of a predetermined burst number per pair. During application of biphasic pulses, such as in H-FIRE, the cathode and anode of an electrode pair are quickly swapped based on the pulsing protocol, so either electrode will experience notable gas formation while being used as the cathode.

For these reasons, pulse delivery strategies that (1) mitigate bubble formation or (2) minimize the risk of arcing due to the presence of bubbles are highly desirable. The methods introduced herein can be used to achieve the latter, and experimental results suggest that the modified waveforms described can prevent mechanical tissue destruction and allow for higher applied potentials prior to the onset of electrical arcing.

### SUMMARY OF THE INVENTION

Pulsed electric fields have been used for biomedical applications for decades. Within the last ten years, the use of bursts of microsecond-duration pulses has become a mainstay of these applications, particularly for tissue ablation and cell permeabilization endpoints. The translation of these burst waveforms to the clinic is underway, but there has not been a study thus far that has sought to introduce a rigorous methodology for constructing such bursts, nor has much attention been paid to the effects of the delays within these burst waveforms. Thus, the methods described herein are intended to allow for precise control over biological outcomes resulting from application of monopolar and bipolar pulsed electric fields including permeabilization, tissue ablation, and nerve excitation. The methods also provide a framework for selecting the temporal infrastructure of bursts of bipolar or monopolar electrical pulses. These methods are to be employed when (1) determining the infrastructure of a burst of bipolar pulses, (2) attempting to manipulate ablation volume without changing the amount of energy applied, or (3) changing the likelihood of stimulating excitable cells during treatments with pulsed electric fields.

More specifically, methods of pulse delivery that can be used to mitigate bubble formation and/or minimize the risk of arcing, such as due to the presence of bubbles, and/or mitigate muscle stimulation are described herein, including the following Aspects.
A method of applying electrical energy to tissue, the method comprising: administering to a tissue a series of bursts of bipolar electrical pulses, wherein each bipolar electrical pulse comprises a positive phase and a negative phase, wherein each phase has a duration of 250 ns to 250 μs, with or without a delay between the positive and negative phases; wherein one or more or each bipolar electrical pulse is separated by a delay lasting up to 1 ms; wherein the bipolar electrical pulses are repeated until a total energized time of 1 μs to 500 μs is achieved; wherein one or more of the bursts has a burst width of at least 1 μs; wherein the series of bursts is administered for a total time of 250 ms to 10 seconds; and wherein the bipolar electrical pulses are applied at a voltage ranging from 500 V to 10 kV.
Aspect 2 is the method of Aspect 1, wherein the series of bursts is administered in a manner to provide reversible electroporation or irreversible electroporation.
Aspect 3 is the method of Aspect 1 or 2, wherein where cardiac synchronization is desired one or more of the bursts has a burst width of 10 μs to 300 ms.
Aspect 4 is the method of any of Aspects 1-3, wherein each phase has a duration of 250 ns to 10 μs and the bipolar electrical pulses are applied at a voltage ranging from 1-5 kV.
Aspect 5 is the method of any of Aspects 1-4, wherein one or more of the bursts has a burst width of 100 μs.
Aspect 6 is the method of any of Aspects 1-5, wherein there is a delay between one or more of the positive phases and the negative phase.
Aspect 7 is the method of any of Aspects 1-6, wherein the delay between the positive phase and the negative phase is shorter than the delay between one or more of the bipolar electrical pulses or between each bipolar electrical pulse, or the delay between the positive phase and the negative phase is longer than the delay between one or more of the bipolar electrical pulses or between each bipolar electrical pulse.
Aspect 8 is a method for applying electrical energy to tissue, the method comprising: administering to a tissue a series of bursts of monopolar electrical pulses, wherein each burst comprises a series of positive and/or negative pulses; wherein one or more or each of the positive or negative pulses has a duration of 250 ns to 250 μs; wherein one or more or each of the positive or negative pulses is separated by a delay of 10 μs to 100 ms; wherein the positive or negative pulses are repeated until a total energized time of 1 μs to 500 μs is achieved; wherein one or more of the bursts has a burst width of at least 10.5 μs; wherein the burst interval of at least two of the bursts is between 250 ms and 10 seconds; and wherein the positive or negative pulses are applied at a voltage ranging from 25 V to 10 kV.
Aspect 9 is the method Aspect 8, wherein the series of bursts is administered to provide reversible electroporation or irreversible electroporation.
Aspect 10 is the method of Aspect 9, wherein one or more of the positive or negative pulses are applied at a voltage ranging from 500 V to 5 kV.
Aspect 11 is the method of Aspect 9 or 10, wherein one or more of the positive or negative pulses or each have a duration of 250 ns to 10 μs.
Aspect 12 is the method any of Aspects 8-11, wherein one or more bursts are administered once every 0.5 s to 3 s.
Aspect 13 is the method of any of Aspects 8-12, wherein at least one of the bursts has a burst width of 100 μs.
Aspect 14 is a method of applying electrical energy to tissue, the method comprising: applying one or more burst of a plurality of electrical pulses to a tissue; wherein one or more of the bursts comprises energy with positive phases and/or negative phases; wherein one or more parameters of the burst are selected such that the burst is capable of causing electroporation of the tissue in a manner that avoids, prevents, minimizes or suppresses neural excitation, bubble formation, and/or Joule heating.
Aspect 15 is the method of Aspect 14, wherein one or more of the parameters is that a time between one of the pulses and another of the pulses is longer than a time between one of the positive phases and one of the negative phases.
Aspect 16 is the method of Aspect 14 or 15, wherein the time between one of the positive phases and one of the negative phases comprises an interphase delay of up to 5 μs, such as ranging from 0 to 5 μs.
Aspect 17 is the method of any of Aspects 14-16, wherein the time between one of the electrical pulses and another of the electrical pulses comprises an interpulse delay of up to 1 second, such as in the range of 10 μs to 1 second.
Aspect 18 is the method of any of Aspects 14-17, wherein the interpulse delay is up to 1,000 times longer than, such as from 5 to 1,000 times longer than, an interphase delay of the electrical pulses.
Aspect 19 is the method of any of Aspects 14-18, wherein one or more of the bursts has a total energized time of up to 1 second, such as from 1 μs to 1 s.
Aspect 20 is the method of any of Aspects 14-19, wherein one or more of the pulses, one or more of the positive phases, and/or one or more of the negative phases have a pulse length of up to 10 μs, such as up to 1 μs, up to 2 μs, up to 5 μs, from 5-10 μs, or from 1-10 μs.

### BRIEF DESCRIPTION OF THE DRAWINGS

The accompanying drawings illustrate certain aspects of implementations of the present disclosure, and should not be construed as limiting. Together with the written description the drawings serve to explain certain principles of the disclosure.

FIGS. 1A-E are graphs showing representative waveforms according to embodiments of the invention of an IRE pulse (1A), a symmetric H-FIRE burst (1B) (i.e., where delay d₁=delay d₂), an asymmetric H-FIRE burst (1C) (i.e., where delay d₂ is longer than delay d₁), a monopolar pulsing scheme comprising positive and negative pulses and where delays d₁ and d₂ are of different lengths (1D); and a monopolar pulsing scheme comprising positive monopolar pulses separated by delays d₁ and d₂, which are of different lengths.

FIGS. 2A-B are photographs showing 2,3,5-triphenyltetrazolium chloride (TTC) stained porcine liver after generating ex vivo ablations with a conventional H-FIRE waveform (2A) and a modified waveform with a long interpulse delay (2B).

FIGS. 3A-B are graphs showing the ablation sizes (3A) and ablation widths (3B) for conventional H-FIRE waveforms and modified waveforms with long interpulse delays according to embodiments of the invention.

FIG. 4A is an illustration showing the experimental setup to determine the maximum voltage that can be applied prior to the onset of arcing.

FIG. 4B is a graph of the maximum voltage that can be applied prior to the onset of arcing for conventional H-FIRE waveforms and modified waveforms with long interpulse delays.

FIG. 5 is an illustration of an equivalent circuit model for a myelinated nerve fiber.

FIG. 6 is a graph showing the strength/duration curve for myelinated nerve fiber in response to single monopolar and bipolar pulsed fields.

FIGS. 7A-B are graphs showing the threshold electric fields required for stimulation for 1 μs pulse bursts (7A) and 5 μs pulse bursts (7B).

FIGS. 8A-J are confocal images of ablations produced by each combination of pulse width (Tₚ) and interpulse delay (d₂).

FIGS. 8K-L are graphs showing the ablation areas (8K) and lethal thresholds (8L) produced by each combination of pulse width (Tₚ) and interpulse delay (d₂).

FIGS. 9A-D are schematic diagrams of electric field contours for bursts with constitutive pulse widths of 1 μs (9A, 9C) and 5 μs (9B, 9D), and interpulse delays of 0.1 μs (9A-B) and 1,000 μs (9C-D).

FIGS. 9E-F are graphs showing the percent difference in exposure of tissue to therapeutic electric fields for each variable delay waveform versus a symmetric burst with interphase delay/interpulse delay set to 1 μs and pulse widths of 1 μs (9E) and 5 μs (9F).

FIGS. 10A-D are schematic diagrams of thermal damage (%) contours for 1 μs pulse burst (10A, 10C) and 5 μs pulse burst (10B, 10D) waveforms with interpulse delays of 0.1 μs (10A-B) and 1,000 μs (10C-D).

FIGS. 11A-C are graphs showing the areas of excitation, IRE, and thermal damage for 1 μs pulse bursts (11A) and 5 μs pulse bursts (11B) as a function of interpulse delay (d₂), and for a traditional IRE protocol (11C).

FIGS. 11D-E are graphs showing the normalized R_{eff} as a function of interpulse delay (d₂) for 1 μs bursts (solid) and 5 μs bursts (dashed) (11D) and for a traditional IRE protocol (11E).

FIGS. 12A-B are graphs showing the ablation areas and maximum voltages that can be applied while maintaining the same area of excitation as with symmetric interpulse delays for bursts with 1 μs (12A) and 5 μs (12B) constitutive pulse widths.

FIGS. 13A-B are graphs showing the external potential at each of six nodes in the model arising from bursts with 5 μs constitutive pulse widths with intraphase delay (d₁) set to 5 μs and interpulse delay (d₂) set to 5 μs (13A) and 100 μs (13B).

FIGS. 13C-D are graphs showing the normalized membrane potentials of the waveforms in FIGS. 13A-B.

FIG. 14 is a graph showing the effect of delay within a burst of monopolar pulses on the resulting threshold for nerve excitation.

FIGS. 15A-D are graphs showing representative depictions of the transmembrane potentials and the electric field over time for a burst of bipolar pulses with no delays (15A, 15C); and with an intraphase delay of 1 μs and an interpulse delay of 10 μs (15B, 15D).

FIGS. 16A-C are graphs showing effects of modifying delays on the resulting characteristic frequency (16A), baseline conductivity (16B), and fold change in conductivity (16C).

### DETAILED DESCRIPTION OF VARIOUS EMBODIMENTS OF THE INVENTION

Reference will now be made in detail to various exemplary embodiments of the invention. It is to be understood that the following discussion of exemplary embodiments is not intended as a limitation on the invention. Rather the following discussion is provided to give the reader a more detailed understanding of certain aspects and features of the invention.

Throughout the present teachings, any and all of the features and/or components disclosed or suggested herein, explicitly or implicitly, may be practiced and/or implemented in any combination, whenever and wherever appropriate as understood by one of ordinary skill in the art. The various features and/or components disclosed herein are all illustrative for the underlying concepts, and thus are non-limiting to their actual descriptions. Any means for achieving substantially the same functions are considered as foreseeable alternatives and equivalents, and are thus fully described in writing and fully enabled. The various examples, illustrations, and embodiments described herein are by no means, in any degree or extent, limiting the broadest scopes of the inventions presented herein or in any future applications claiming priority to the instant application.

The enclosed methods describe modifications to pulsed electric field waveforms that may enhance their biological and clinical outcomes while mitigating unwanted effects such as nerve stimulation, bubble formation, and/or electrical arcing. Traditionally, bursts of bipolar pulses were developed with relatively short, symmetric delays between pulses in the waveform simply to mitigate assisted discharge between subsequent pulses of alternating polarity and to provide a protective barrier for MOSFET switching. Translation of this approach to monopolar pulses has also been described with the same rationale. The methods introduced herein allow the user to select the delays within these delivery strategies such that the total energy delivered is equivalent, while the extent of muscle stimulation, bubble formation, and/or electrical arcing are reduced and biophysical effects are maintained or magnified.

The methods described are applicable for applications in which pulsed electric fields are used. These include treatments such as electrochemotherapy, electrogene transfer, electropermeabilization, non-thermal tissue ablation, etc. The methods introduced herein allow the user of these technologies to employ a pulse regimen which yields the abovementioned outcomes with reduced stimulation of excitable cells, bubble formation, and electrical arcing. Thus, these methods may allow parties using pulsed electric fields to employ lower dosages of neuromuscular blocking agent and, in some cases, employ these pulses without the need for nerve paralytics.

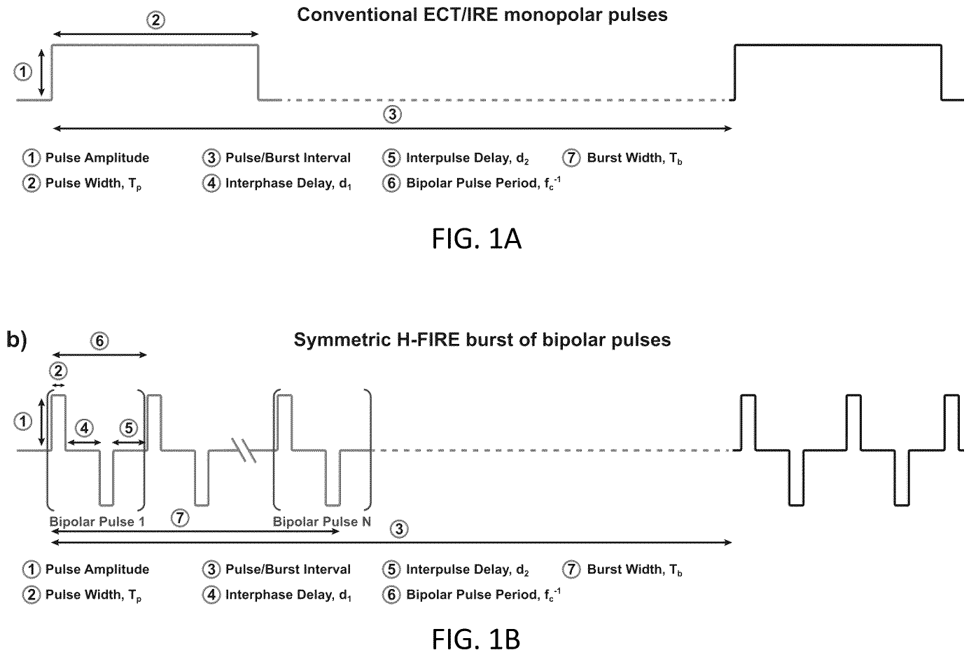

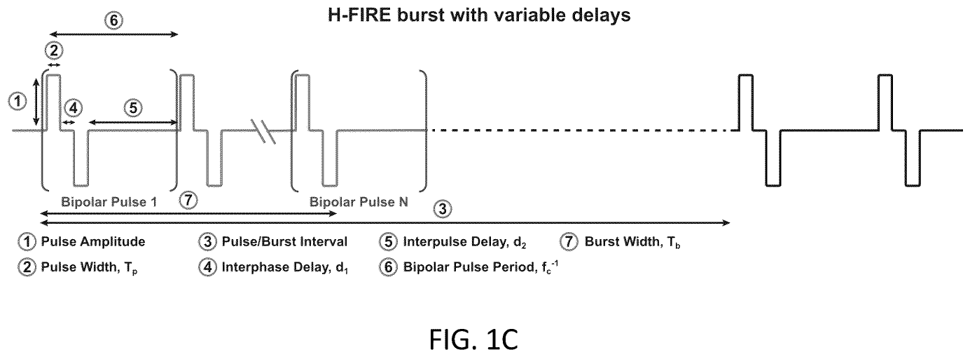

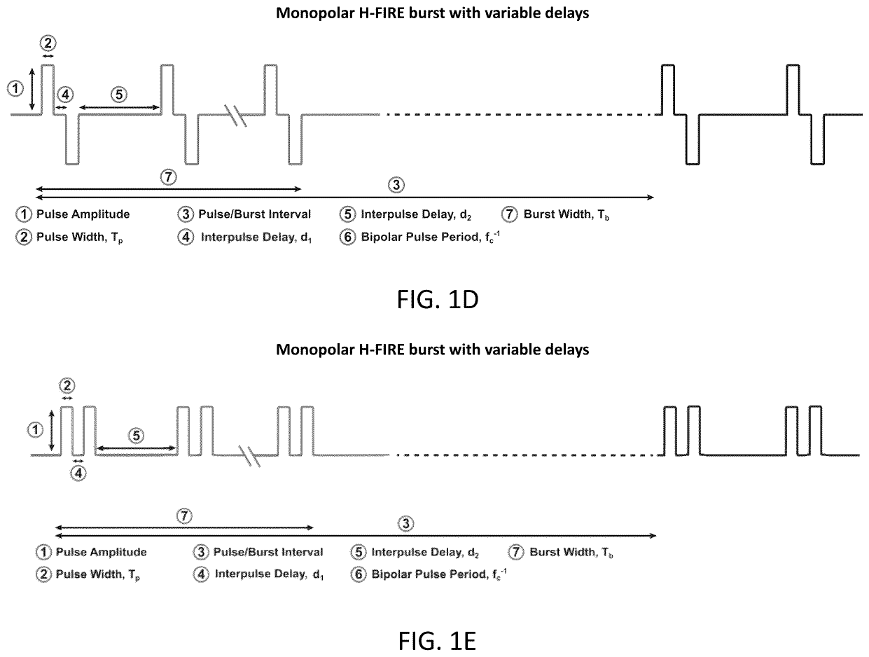

According to embodiments of the invention, the length of the delays (e.g., delays d₁ and d₂) can be selected to minimize bubble formation and/or any electrical arcing. In traditional IRE (e.g., FIG. 1A), the pulses and delays are typically longer as compared with traditional H-FIRE (e.g., FIG. 1B). For example, with traditional H-FIRE protocols, interphase delays (e.g., d₁) and interpulse delays (e.g., d₂) typically range from 1-5 μs. Embodiments of the present invention comprise administration of H-FIRE bursts with extended interpulse delays (e.g., d₂) being longer than the interphase (e.g., d₁) delays (FIG. 1C). “Extended” here means longer than the traditional H-FIRE type delays of 1-5 μs. According Equation 2, the ideal length of this delay will depend on one or more of pulse width and on time, as well as similar factors/parameters, but in embodiments, the interpulse delay (e.g., d₂) is in the range of tens to a few hundred microseconds, such as about 10 μs, 20 μs, 30 μs, 50 μs, 75 μs, 100 μs, 125 μs, 150 μs, 200 μs, 250 μs, 300 μs, 500 μs, 750 μs, or 1,000 μs. It is desirable for this delay to be as long as possible and according to various embodiments “extending” it out to 1 ms is feasible. According to the Examples, with a 1 ms interpulse delay (e.g., d₂), nerve excitation thresholds are increased ˜60% for bursts with 5 μs pulse widths, and can be more than doubled for bursts with 1 μs pulse widths. However, this also depends on how long the interphase delay (e.g., d₁) is, as well as the pulse width and on time.

In embodiments, the plurality of electrical pulses can have a pulsing scheme that incorporates one or more delays (d₁ and/or d₂). Bipolar pulse schemes or monopolar pulse schemes may be described with an X-X-X-X convention, which can include any one or more of a pulsing protocol of the following formats:
Bipolar pulsing: positive phase-delay (d₁)-negative phase-delay (d₂);
Bipolar pulsing: negative phase-delay (d₁)-positive phase-delay (d₂);
Monopolar pulsing: positive pulse-delay (d₁)-negative pulse-delay (d₂);
Monopolar pulsing: negative pulse-delay (d₁)-positive pulse-delay (d₂);
Monopolar pulsing: positive pulse-delay (d₁)-positive pulse-delay (d₂);
Monopolar pulsing: negative pulse-delay (d₁)-negative pulse-delay (d₂).

For example, a monopolar pulse scheme of 5-2-5-250 provides for a 5 μs monopolar pulse (positive or negative), followed by a 2 μs delay (d₁), followed by a 5 μs monopolar pulse (positive or negative), followed by a 250 μs delay (d₂). Additionally, a bipolar pulse scheme of 5-2-5-250 provides for a 5 μs positive (or negative) phase of a bipolar pulse, followed by a 2 μs delay (d₁), a 5 μs negative (or positive) phase of a bipolar pulse, and a 250 μs delay (d₂).

### Definitions

The term “pulse” refers to an electrical signal with a single phase (monopolar, unipolar) or more than one phase (bi-polar). If bi-polar, there can be a delay between phases or the switch between phases/polarity can be immediate (no delay).

The term “interphase delay” refers to a period of time where no energy is applied/delivered/administered between one phase and another phase. An interphase delay can occur between phases of a bi-polar pulse (e.g., a delay between the positive portion of a bipolar pulse and the negative portion of the bipolar pulse or a delay between the negative portion of a bipolar pulse and the positive portion of the bipolar pulse). An interphase delay can occur between a negative portion of a bipolar pulse and a positive portion of another bipolar pulse, or a delay between a positive portion of a bipolar pulse and a negative portion of another bipolar pulse, or a delay between a positive portion of a bipolar pulse and a positive portion of another bipolar pulse, a delay between a negative portion of a bipolar pulse and a negative portion of another bipolar pulse. An interphase delay can be a delay that occurs between monopolar pulses, such as between monopolar pulses of the same or different polarity (e.g., a delay between a positive monopolar pulse and a negative monopolar pulse, or a delay between a negative monopolar pulse and a positive monopolar pulse, or a delay between a positive monopolar pulse and another positive monopolar pulse, or a delay between a negative monopolar pulse and another negative monopolar pulse). An interphase delay can occur between one phase of a bipolar pulse and a monopolar pulse or between a monopolar pulse and one phase of a bipolar pulse.

The term “interpulse delay” refers to a period of time where no energy is applied between one pulse and another pulse, or between one pulse and a set of pulses, or between a set of pulses and another pulse, or between one bipolar pulse and a set of bipolar pulses, or between one bipolar pulse and another bipolar pulse, or between a set of bipolar pulses and a bipolar pulse, or between a monopolar pulse and another monopolar pulse, between a monopolar pulse and a set of monopolar pulses, or between a set of monopolar pulses and a monopolar pulse, or between a positive monopolar pulse and another positive monopolar pulse, or between a negative monopolar pulse and another negative monopolar pulse, or between a positive monopolar pulse and a negative monopolar pulse, or between a negative monopolar pulse and a positive monopolar pulse.

In some cases, a delay between pulses, or an interpulse delay, may also be referred to as an interphase delay, such as where the delay occurs between phases of a pulse or pulses. For example, an interpulse delay that is also an interphase delay may occur between a positive monopolar pulse and a negative monopolar pulse, or between a negative monopolar pulse and a positive monopolar pulse, or between a positive monopolar pulse and a positive monopolar pulse, or between a negative monopolar pulse and a negative monopolar pulse, or between one phase of a bipolar pulse (positive or negative) and one phase of another bipolar pulse (positive or negative), or between a monopolar pulse of either phase and either phase of a bipolar pulse.

Delays in the context of this disclosure can be expressed as d₁ or d₂ delays. The term “d₁” refers to a delay in a pulsing scheme, typically a first type of delay in a pulsing protocol. In embodiments, a burst of electrical pulses can comprise none, one or more, or multiple d₁ delays, which in some cases may be referred to as an interphase delay, an interpulse delay, an intraphase delay or an intrapulse delay. The term “d₂” refers to another delay in a pulsing scheme. In embodiments, a burst of electrical pulses can comprise none, one or more, or multiple d₂ delays, which in some cases may be referred to as an interpulse delay or an interphase delay or an intrapulse delay or intraphase delay.

The terms “asymmetric delay” or “variable delay” refer to delays that are not equal in length, e.g., one delay is longer than another. In embodiments, a monopolar pulse scheme may have asymmetric interpulse delays. In embodiments, an interphase delay can be a different length than the interpulse delay, such as one or more interphase delay that is shorter than one or more interpulse delay, or one or more interphase delay that is longer than one or more interpulse delay. In embodiments, an interphase delay can be longer than an interpulse delay within a burst. Also in embodiments, an interphase delay can be variable/asymmetric (e.g., have a different length) as compared with another interphase delay within a burst and/or an interpulse delay can be variable or asymmetric with respect to another interpulse delay within a burst of pulses, or as compared with delays of other bursts, such as in a series of bursts.

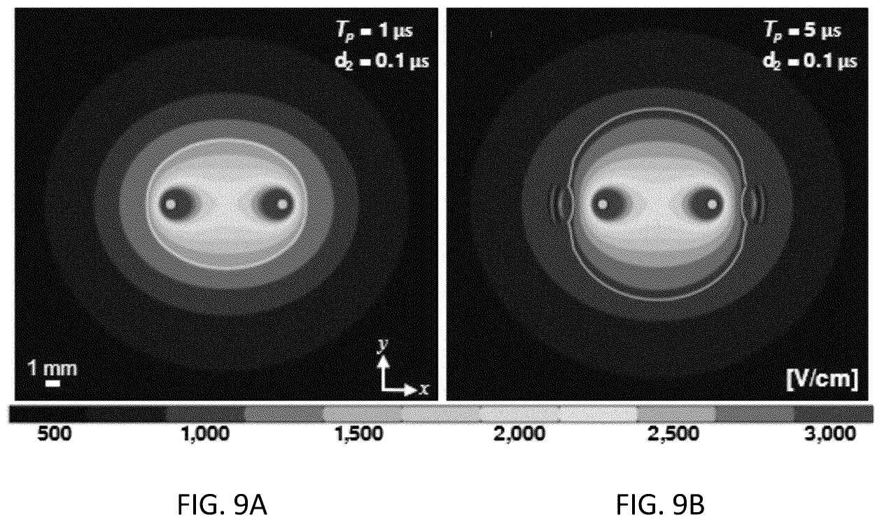

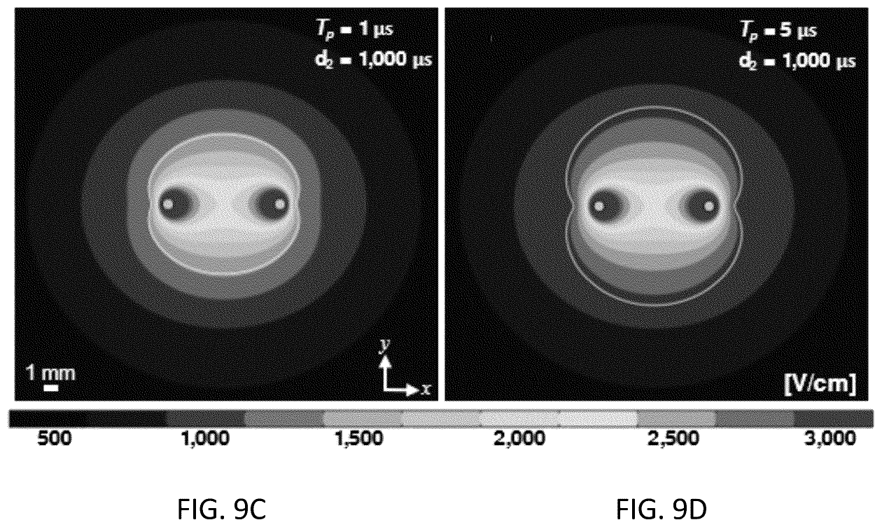

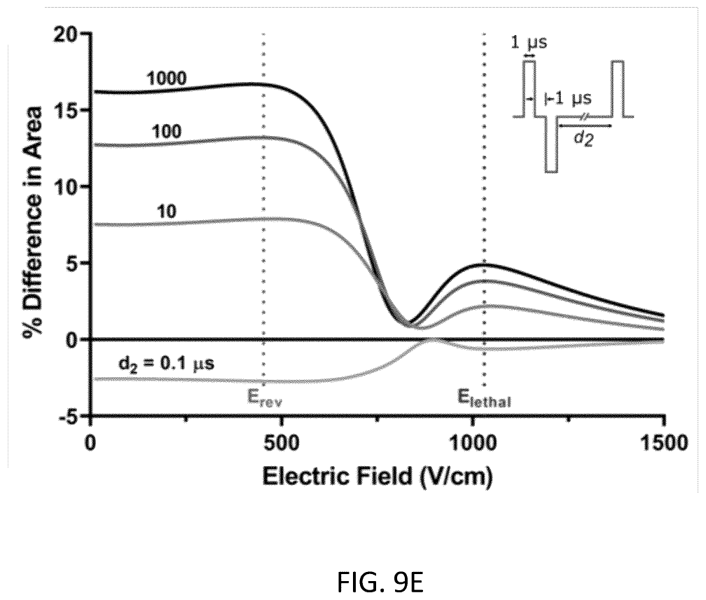

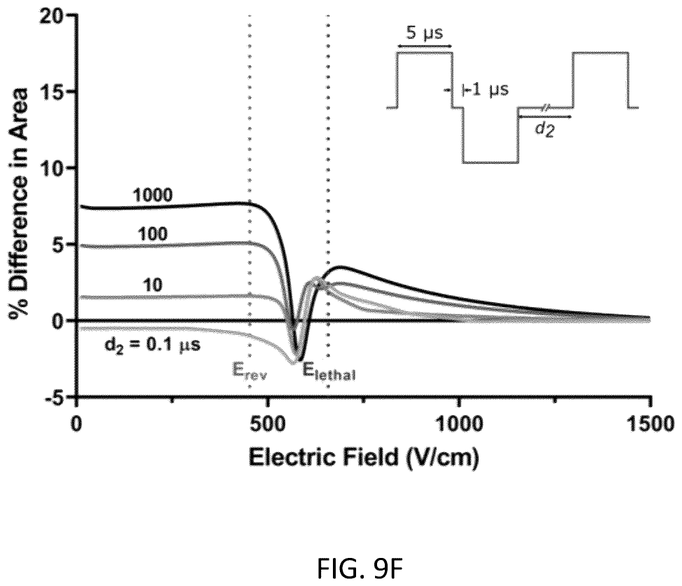

For example, one or more interphase delay and/or one or more interpulse delay, such as within a burst of pulses, can range in length of up to 1 ms, such as from 0.5 μs up to and including 1 ms, such as from 2 μs to 500 μs, or from 5 μs to 900 μs, or from 10 μs to 100 μs, or from 15 μs to 700 μs, or from 50 μs to 300 μs, or from 75 μs to 750 μs, or from 4 μs to 150 μs, or from 8 μs to 350 μs, or from 30 μs to 250 μs, and so on. In embodiments, the interphase or interpulse delay is up to about 0.5 μs, 1 μs, 1.5 μs, 2 μs, 2.5 μs, 3 μs, 4 μs, 5 μs, 6 μs, 7 μs, 8 μs, 9 μs, or 10 μs. In embodiments, the interpulse delay is up to about 10,000 μs, such as up to about 1000 μs, 1500 μs, 2000 μs, 2500 μs, 3000 μs, 3500 μs, 4000 μs, 4500 μs, 5000 μs, 5500 μs, 6000 μs, 6500 μs, 7000 μs, 7500 μs, 8000 μs, 8500 μs, 9000 μs, or 9500 μs. Indeed, any interphase and/or interpulse delay is feasible, including any delay that falls within any of these ranges, or any range derived from one or more of these endpoints. An interphase delay can be 1, 2, 3, 4, 5, 6, 7, 8, 9, or 10 times, or even up to 100 times the length of an interpulse delay, such as within a burst of pulses, and vice versa. Essentially the longer the interpulse delay (up to 1 ms), the lower the characteristic frequency of the burst, and thus, the lower the “baseline” electrical conductivity. Assuming the conductivity due to electroporation saturates to a relatively constant final value, lowering the initial conductivity will provide a larger change in conductivity, which helps the field propagate further and increase ablation size. How this affects the field distribution is shown in FIGS. 9A-F.

In embodiments, a bipolar pulse can comprise a positive portion and a negative portion of a pulse, any delay d₁ and any delay d₂.

In embodiments, a burst of monopolar pulses can comprise a positive pulse, a delay d₁, a negative pulse, and a delay d₂; a negative pulse, a delay d₁, a positive pulse, and a delay d₂; a positive pulse, delay d₁, a positive pulse, and delay d₂; or a negative pulse, delay d₁, a negative pulse, and a delay d₂.

The term “burst” refers to a set of pulses, a group of pulses, or a pulse group.

The term “burst width” refers to the pulses/phases and any inter-pulse/phase delay.

The term “burst interval” refers to the pulses/phases and any inter-pulse/phase delay plus any inter-burst delay.

The term “thermal damage” refers to damage to a treated tissue caused by an increase in temperature which results in death of the tissue and/or denaturing of proteins.

From a thermal perspective, if baseline conductivity is lower due to the lower characteristic frequency, that means less current and reduced thermal effects. By extending the delay, it is expected that any products of electrolysis (such as hydrogen bubbles) will have more time to diffuse away from the electrodes during energy delivery. Thus, the fewer bubbles immediately adjacent to the probe will mean less arcing.

In embodiments, electroporation is administered by way of a plurality of electrical pulses, wherein one or more pulses of the plurality of electrical pulses has a pulse length in the picosecond to microsecond range, such as in the nanosecond to microsecond range, including from 1 picosecond to below 10 microseconds, or from 1 picosecond to 1 microsecond, or below 1 microsecond, or from at least 0.1 microsecond up to 5 microseconds, or from 0.5 microseconds up to 2 microseconds or up to 10 microseconds, such as up to 100 ns, 250 ns, 500 ns, 1 μs, 2 μs, 5 μs, 10 μs, 15 μs, 20 μs, 25 μs, 40 μs, 50 μs, 60 μs, 75 μs, 85 μs, 100 μs, 110 μs, 125 μs, or 150 μs, or even up to about 200 μs or any range in between any of these ranges or endpoints, including as endpoints any number encompassed thereby, such as a high-frequency irreversible electroporation burst scheme of pulse width and intra-phase delay ranging from 0.1 μs to 10 ms and an inter-pulse delay ranging from 0.1 μs to 1 s.

In embodiments, the plurality of electrical pulses are administered at a voltage in the range of 0 V to about 15,000 V, such as above 0 V or 1 V up to about 1,000 V, for example, up to about 10 V, 15 V, 20 V, 30 V, 50 V, 60 V, 75 V, 100 V, 150 V, 200 V, 225 V, 250 V, 300 V, 350 V, 375 V, 400 V, 425 V, 450 V, 500 V, 600 V, 650 V, 800 V, or 900 V. In embodiments, the plurality of electrical pulses are administered at a voltage in the range of above 0 V or 1 V up to about 15 kV, such as up to about 1.2 kV, 1.5 kV, 1.75 kV, 2 kV, 2.2 kV, 2.5 kV, 2.7 kV, 3 kV, 3.5 kV, 4 kV, 4.5 kV, 5 kV, 5.5 kV, 6 kV, 7 kV, 7.5 kV, 8 kV, 9 kV, 10 kV, 11 kV, 12 kV, 13 kV, or 14 kV. In embodiments, the amplitudes of the monopolar pulses or each phase of the bipolar pulses are symmetrical. In other embodiments, the amplitudes are asymmetrical. In embodiments, a burst of pulses or a series of bursts can comprise only symmetric pulses/phases, or only asymmetric pulses/phases, or a combination of symmetric and asymmetric pulses/phases. In this context, asymmetric amplitude refers to the voltage applied for one pulse/phase is different than the voltage applied for another pulse/phase, such as one pulse/phase having a higher amplitude than another pulse/phase, or one pulse/phase having a lower amplitude than another pulse/phase.

In embodiments, the number of pulses is administered and/or a total number of pulses per burst, ranges from 1-5,000 pulses, such as from at least 1 up to 3,000 pulses, or at least 2 up to 2,000 pulses, or at least 5 up to 1,000 pulses, or at least 10 up to 500 pulses, or from 10 to 100 pulses, such as from 20 to 75 pulses, or from 30 to 50 pulses, such as 1, 5, 10, 15, 20, 25, 30, 35, 40, 45, 50, 60, 70, or 90 pulses, or the total number of pulses and/or bursts can range from 1 to 5,000 pulses/bursts, such as from at least 1 up to 3,000 pulses/bursts, or at least 2 up to 2,000 pulses/bursts, or at least 5 up to 1,000 pulses and/or bursts, or at least 10 up to 500 pulses/bursts, or from 10 to 100 pulses/bursts, such as from 20 to 75 pulses/bursts, or from 30 to 50 pulses/bursts, such as 1, 5, 10, 15, 20, 25, 30, 35, 40, 45, 50, 60, 70, or 90 pulses/bursts, or any range in between any of these ranges or endpoints, including as endpoints any number encompassed thereby.

In embodiments, the pulse rate can have a frequency in the range of about 0 Hz to 100 MHz, such as from above 0 Hz or 1 Hz up to 100 MHz, such as from 2 Hz to 100 Hz, or from 3 Hz to 80 Hz, or from 4 Hz to 75 Hz, or from 15 Hz to 80 Hz, or from 20 Hz up to 60 Hz, or from 25 Hz to 33 Hz, or from 30 Hz to 55 Hz, or from 35 Hz to 40 Hz, or from 28 Hz to 52 Hz, or a frequency ranging from 100 Hz to 100 MHz, such as in the Hz range from 100 Hz or 1 Hz up to 100 Hz, or from 2 Hz to 100 Hz, or from 3 Hz to 80 Hz, or from 4 Hz to 75 Hz, or from 15 Hz to 80 Hz, or from 20 Hz to 60 Hz, or from 25 Hz to 33 Hz, or from 30 Hz to 55 Hz, or from 35 Hz to 40 Hz, or from 28 Hz to 52 Hz, or a frequency in the kHz or MHz range, such as from 1 kHz to 10 kHz, or from 2 kHz to 8 kHz, or from 3 kHz to 5 kHz, or from 4 kHz 9 kHz, or from 7 kHz to 15 kHz, or from 6 kHz to 20 kHz, or from 12 kHz to 30 kHz, or from 25 kHz to 40 kHz, or from 5 kHz to 55 kHz, or from 50 kHz to 2 MHz, including any range in between, such as from 10-25 kHz, or from 15-40 kHz, or from 20-50 kHz, or from 75 kHz to 150 kHz, or from 100 kHz to 175 kHz, or from 200 kHz to 250 kHz, or from 225 kHz to 500 kHz, or from 250 kHz to 750 kHz, or from 500 kHz to 1 MHz, or any range in between any of these ranges or endpoints, including as endpoints any number encompassed thereby. In embodiments, the pulse rate can be the same or different for different bursts of the series of bursts.

In embodiments, the shape of the electrical pulses delivered can be any desired waveform, including square, triangular, trapezoidal, exponential decay, sawtooth, sinusoidal, and/or such waveforms comprising one or more pulses of alternating polarity.

The present invention is further illustrated by the following Examples intended to demonstrate certain principles and features of the invention and not limit the scope of any claim.

### Example 1

Porcine livers were harvested from a local abattoir and anastomosed to a mechanical perfusion system as previously described (Bhonsle, S. et al., “Characterization of Irreversible Electroporation Ablation with a Validated Perfused Organ Model,” J. Vasc. Interv. Radiol., 2016, 27(12), 1913-1922). An internally perfused single applicator (exposure=7.5 mm, Ø=1.65 mm) was used as the local monopolar source, with a surface electrode (SA˜300 cm²) placed under the liver as the indifferent electrode. Both were made of stainless steel. A custom high-voltage generator was used to deliver electrical pulses. Standard H-FIRE bursts consisted of 100 μs of energized time, with the pulse width (Tₚ) set to 5 μs and the interphase delay (d₁) and the interpulse delay (d₂) both set to 5 μs (burst width (T_{b})=195 μs). Modified H-FIRE bursts (as proposed herein) were identical except d′ was set to 2 μs and de was set to 250 μs (T_{b}=2,370 μs). In each treatment, sets of 25 bursts were delivered at a rate of 1 Hz, with each set separated by a 30 second delay until 400 total bursts were delivered. Applied voltage was set to 4 kV.

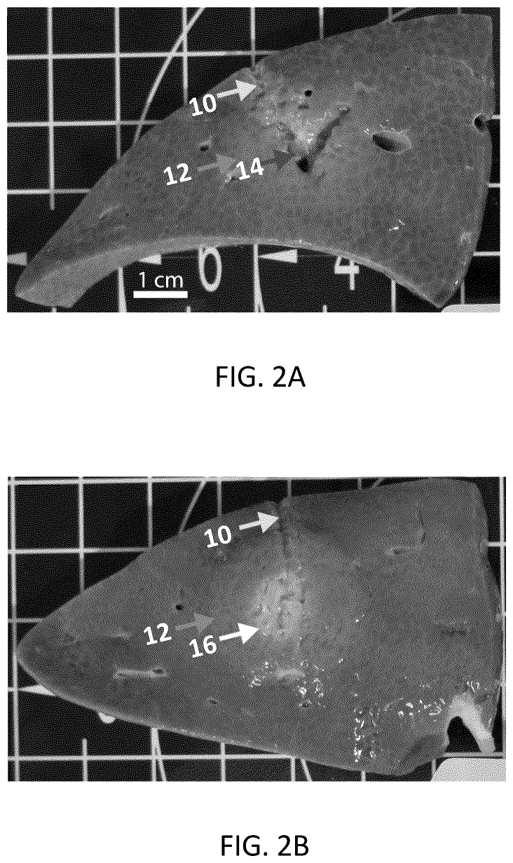

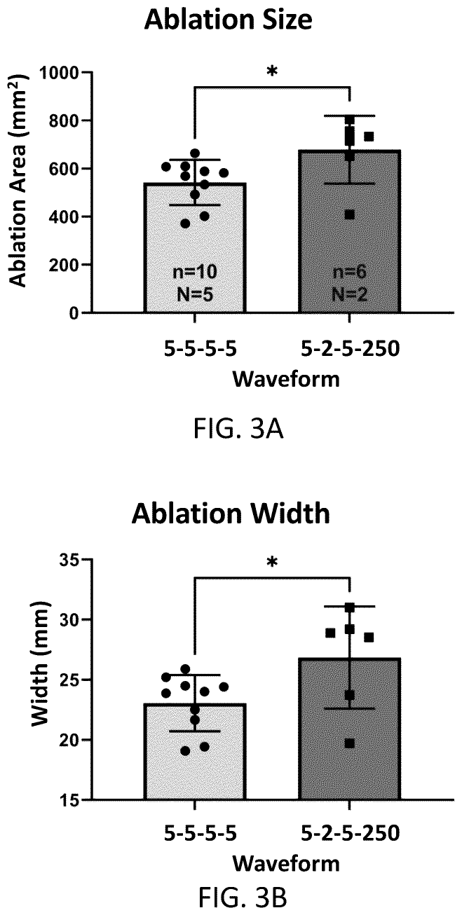

FIGS. 2A-B show 2,3,5-triphenyltetrazolium chloride (TTC) stained porcine liver after generating ex vivo ablations with a conventional H-FIRE waveform (5-5-5-5) (FIG. 2A) and a modified waveform with a longer interpulse delay (5-2-5-250) (FIG. 2B). In the figures, the electrode insertion tract 10, the ablation boundary 12, the area in which tissue has been physically destroyed 14, and the area undergoing thermal damage 16 are indicated with arrows. Ablation areas arising from traditional H-FIRE treatments (FIG. 2A) are not as well demarcated or spherical as those created with modified waveforms (FIG. 2B). Additionally, the geometry of ablated and thermally damaged tissue with modified waveforms is consistently ellipsoidal, as predicted by numerical models. Traditional H-FIRE waveforms also resulted in the formation of “voids” where it is presumed that tissue was mechanically destroyed due to rapid gas bubble formation and cavitation due to the structure of the waveform. The long interpulse delays may allow gas bubbles time to migrate away from electrode surfaces or dissolve prior to onset of the subsequent bipolar pulse, protecting local tissue from mechanical destruction. Overall, modified waveforms (5-2-5-250) also produced larger ablation areas (p=0.0349) (FIG. 3A) and ablation widths (p=0.036) (radial diameter, FIG. 3B) as compared to traditional waveforms (5-5-5-5). This suggests these waveforms are comparably efficient or even more efficient at generating ablations as compared to traditional waveforms with short, symmetric delays.

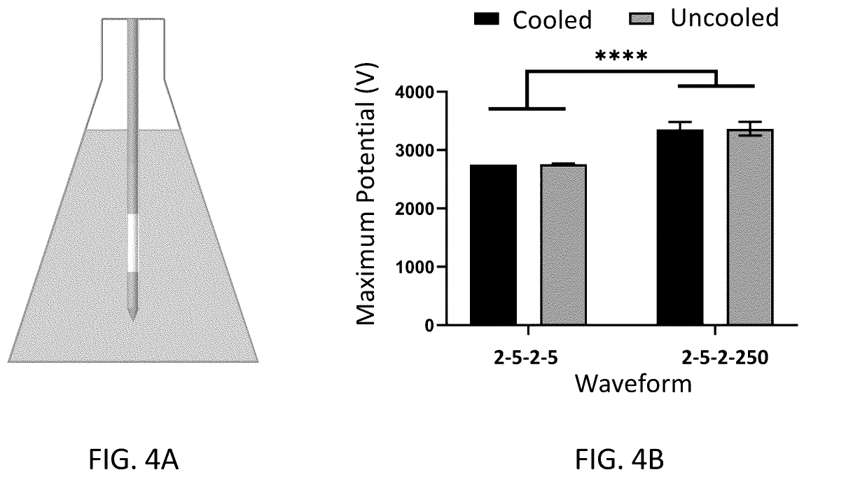

To build on these results, a second experiment was designed to determine if modified waveforms would allow for increased applied potentials with certain probe geometries. An agar (1% w/v) based tissue phantom was constructed in a 250 mL Erlenmeyer flask containing 0.1% NaCl (σ˜0.2 S/m) to mimic the electrical and physical properties of liver tissue. A standard bipolar probe (AngioDynamics, Inc.) was inserted into the agar such that it was centered vertically and radially (FIG. 4A). A custom high-voltage generator was used to deliver electrical pulses. Standard H-FIRE bursts consisted of 100 μs of energized time, with Tₚ set to 2 μs and d₁ and de both set to 5 μs (T_{b}=345 μs). Modified H-FIRE bursts (as proposed herein) were identical except de was set to 250 μs (T_{b}=6,225 μs). Initially, the voltage was set to 2.25 kV and 5 bursts were delivered, then after a 5 second delay the voltage was increased by 250 V and 5 more bursts delivered. This process was repeated until electrical arcing was observed visually and confirmed by current analysis showing a rapid spike in current and generator shutdown. Once arcing was detected, voltage was decreased by 100 V and pulsing was attempted again. The maximum voltage capable of delivering all 5 bursts for a given waveform was recorded.

Results were repeated with a standard solid bipolar probe and in the presence of internal probe cooling (FIG. 4B). Internal electrode cooling was found to have no effect on arcing, but extending d₂ from 5 μs to 250 μs had a major impact. The peak applied potential for the modified 2-5-2-250 was 3361±8 V while that of the standard 2-5-2-5 was 2754±6 V (p<0.0001). Thus, long interpulse delays allowed the applied potential to be increased by approximately 600 V without arcing.

By using the modified waveforms, it is possible to increase the voltage applied clinically, which would increase the size of achievable ablations. Even at the same voltage, modified waveforms are preferred due to their reduced likelihood of electrical arcing. It is proposed that the mitigated arcing can be ascribed to improved bubble dissolution or movement between each bipolar pulse during the interpulse delay, preventing bubble buildup along either electrode and reducing the chance of bubble cavitation. Since bubble dissolution or dissolving takes some finite amount of time (presumably on the order of microseconds), bubble dissolution/dissolving may be more significant with longer delays, such that bubbles are not as prevalent, including in the vicinity of the electrodes. Longer delays may allow for dispersion or dissolving of bubbles that can be present in the vicinity of the electrodes with the traditional H-FIRE waveforms. Likewise, it is anticipated that a similar strategy employed for monophasic bursts would likewise mitigate arcing and maximize the voltage that could be applied.

### Example 2

### Modified SENN Model

To assess the response of a myelinated neuron to a temporally arbitrary electric field, the SENN framework introduced by Reilly et al. (Reilly, J. P. et al., “Sensory Effects of Transient Electrical Stimulation—Evaluation with a Neuroelectric Model,” IEEE Trans. Biomed. Eng., vol. BME-32, no. 12, 1001-1011, 1985). was adopted, but the Frankenhaeuser and Huxley current equations were replaced with Hodgkin-Huxley type formulations computed for mammalian neurons (McIntyre, C. C. et al., “Modeling the excitability of mammalian nerve fibers: Influence of afterpotentials on the recovery cycle,” J. Neurophysiol., vol. 87, no. 2, 995-1006, 2002). This model can provide the nerve fiber response to any transient electric field with known spatial distribution. Table 1 shows parameters used in the modified SENN model.

TABLE 1
Parameters Used in Modified SENN Model

| Quantity | Unit | Value | Ref. |
|---|---|---|---|
| Axoplasmic resistivity, ρᵢ | Ω · cm | 70 | McIntyre, 2002 |
| Membrane capacity, cₘ | μF cm⁻² | 2 | McNeal, 1976 |
| Fiber diameter, D | μm | 1 | Mercadal, 2017 |
| Nodal gap width, W | μm | 1 | Mercadal, 2017 |
| Axon diameter, dₐ | — | 0.7 D | McNeal, 1976 |
| Nodal diameter, dₙ | — | 0.33 D | Mercadal, 2017 |
| Internodal separation, L | mm | 1.15 | Mercadal, 2017 |
| Membrane capacitance | μF | cₘπdₙW | — |
| Membrane conductance | Ω⁻¹ | πdₐ²/(4ρᵢL) | — |
| Rest Potential, Eᵣ | mV | −88 | — |

Because electroporation-based treatments are conventionally performed with needle electrodes, the present inventors chose to model a scenario representing a nerve terminus within the vicinity of the electrodes and in parallel with a given electric field contour, as originally proposed by Mercadal and colleagues (Mercadal, B. et al., “Avoiding nerve stimulation in irreversible electroporation: A numerical modeling study,” Phys. Med. Biol., vol. 62, no. 20, 8060-8079, 2017.). Thus, for each waveform, the electric field required to initiate an action potential in a short nerve segment with 6 nodes of Ranvier exposed to a uniform field was calculated. By assuming a nerve terminus is present at all points in a given domain, these thresholds can be extrapolated to estimate the amount of tissue exposed to fields capable of inducing action potentials irrespective of electrode geometry and stimulus amplitude (Mercadal, 2017). It is worth noting that peripheral motor neurons are excited at lower stimulus magnitudes than skeletal myocytes, so it is not necessary to consider direct stimulation of muscle cells themselves.

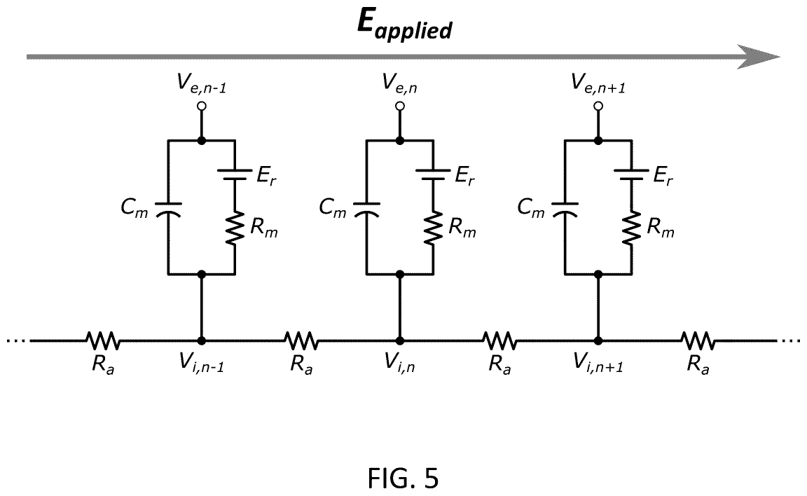

The equivalent circuit employed in this model, originally proposed by McNeal (McNeal, D. R., “Analysis of a Model for Excitation of Myelinated Nerve,” IEEE Trans. Biomed. Eng., vol. BME-23, no. 4, 329-337, 1976.) is given in FIG. 5.

### Determination of Lethal EFTS In Vitro

Lethal thresholds were characterized experimentally for each waveform given in Table III using established methods (Arena, C. B. et al., “A three-dimensional in vitro tumor platform for modeling therapeutic irreversible electroporation,” Biophys. J., vol. 103, no. 9, 2033-2042, 2012.). Briefly, disk-shaped collagen type I hydrogel constructs were fabricated and seeded with hepatocellular carcinoma cell line Hep G2 (ATCC® HB-8065™) at a concentration of 10⁶ cells/mL. Hydrogels were covered in culture media and incubated for 24 hours. Two needle electrodes (4 mm spacing, Ø=0.9 mm) were used to treat gels with the given H-FIRE waveform or IRE in a mobile incubator maintained at 37° C. Voltage (600 V), repetition rate (1 burst/s), energized time (100 μs), and number of bursts (100) were maintained constant. After treatment, media was replenished and cells were incubated for 24 hours prior to live/dead staining with calcein AM and propidium iodide, respectively. Finally, cells were imaged with a confocal microscope and ablation areas were measured in ImageJ (NIH). A 3D numerical model of treatment was constructed (Comsol Multiphysics v5.5) and a function relating electric field strength to area of exposure was created. Ablation areas were used as inputs to the function to estimate lethal EFTs. For more details, see Example 3.

### Realistic In Vivo Ablation Model

To evaluate non-thermal ablation, temperature rise, and thermal damage resulting from treatment with symmetric and variable delay H-FIRE waveforms, a 2-D finite element model representative of a two-needle in vivo ablation was developed. Material properties used in the ablation model are shown in Table II (Hasgall, P. et al., “IT′IS Database for thermal and electromagnetic parameters of biological tissues,” 2018, it is.swiss/database%0A%0A; Duck, F. A., Physical Properties of Tissue: A Comprehensive Reference Book. London: Harcourt Brace Jovanovich, 1990; Cosman, E. R. et al., “Electric and Thermal Field Effects in Tissue Around Radiofrequency Electrodes,” Pain Med., vol. 6, no. 6, 405-424, 2005.).

TABLE II
Material properties used in ablation model.

| Quantity | Unit | Value | Ref. |
|---|---|---|---|
| Density, ρ | kg m⁻³ | 1,079 | Hasgall, 2018 |
| Heat capacity, cₚ | J kg⁻¹ K⁻¹ | 3,540 | Hasgall, 2018 |
| Thermal conductivity, k | W m⁻¹ K⁻¹ | 0.512 | Duck, 1990 |
| Temperature coefficient, α | % K⁻¹ | 2 | Duck, 1990 |
| Blood perfusion, w_{b} | s⁻¹ | 9.27 × 10⁻³ | Cosman, 2005 |
| Blood density, ρ_{b} | kg m⁻³ | 1,050 | Hasgall, 2018 |
| Blood heat capacity, c_{b} | J kg⁻¹ K⁻¹ | 3,617 | Hasgall, 2018 |
| Frequency factor, ξ | s⁻¹ | 2.08 × 10²⁷ | Ibey, 2014 |
| Activation energy, Eₐ | J mol⁻¹ | 1.866 × 10⁵ | Ibey, 2014 |

Tissue electrical conductivity exhibits complex behavior in response to applied electric fields. This behavior is tissue specific and depends on the amplitude and duration of the applied field, as well as the number of pulses. Electroporation-induced conductivity changes can be represented by a sigmoidal curve that: (a) begins at a baseline conductivity go determined by the characteristic frequency of the waveform; (b) exhibits a transition range related to the reversible and irreversible ablation thresholds of the waveform; and (c) saturates to a final conductivity σ_{f} similar to the tissue's conductivity in the upper end of the β-dispersion frequency range (1 kHz-100 MHz). To consider these dynamic changes, the model introduced by Sel et al. was used (Sel, D. et al., “Sequential finite element model of tissue electropermeabilization,” IEEE Trans. Biomed. Eng., vol. 52, no. 5, 816-827, 2005). Baseline electrical conductivity (go) was determined by evaluating a liver impedance model at the characteristic frequency of each waveform, and electroporated conductivity (σ_{f}) was set to 0.32 S/m by evaluating the model at 10 MHz (Table III).

TABLE III
H-FIRE Waveforms Assessed in 2D Ablation Model

| Tₚ (μs) | d₁ (μs) | d₂ (μs) | f_{c} (kHz) | σ₀ (mS cm⁻¹) | T_{b} (ms) |
|---|---|---|---|---|---|
| 1 | 1 | 0.1 | 322.6 | 1.5 | 0.15 |
| 1 | 1 | 1 | 250 | 1.3 | 0.20 |
| 1 | 1 | 10 | 76.9 | 0.81 | 0.64 |
| 1 | 1 | 100 | 9.71 | 0.53 | 5.1 |
| 1 | 1 | 1,000 | 0.997 | 0.41 | 49 |
| 5 | 1 | 0.1 | 90.1 | 0.84 | 0.11 |
| 5 | 1 | 1 | 83.3 | 0.82 | 0.12 |
| 5 | 1 | 10 | 47.6 | 0.72 | 0.20 |
| 5 | 1 | 100 | 9.01 | 0.53 | 1.0 |
| 5 | 1 | 1,000 | 0.989 | 0.41 | 9.1 |
| 100 | N/A | N/A | 10 | 0.535 | 0.1 |

Results showed no difference in lethal EFT as a function of d₂, so E₁ was set to 1,030 V/cm for all waveforms with 1 μs pulses, and 658 V/cm for waveforms made up of 5 μs pulses—the average across all values of d₂ for either constitutive pulse width. The reversible threshold (E₁) was characterized for the 5-1-5-1 protocol (453 V/cm) and assumed constant regardless of d₂ or Tₚ. Thresholds were also characterized for a conventional IRE protocol to provide a comparison. E, for the IRE protocol was set to 282 V/cm, which was computed by scaling the experimentally determined E₁ (429 V/cm) to the reported ratio of E₀/E₁ (Sel, 2005).

### Modified SENN Model Behavior and Validation

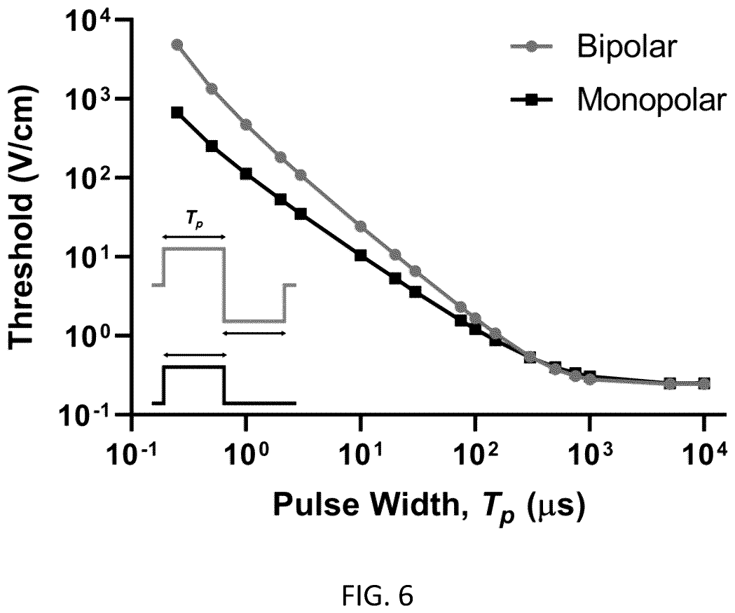

FIG. 6 gives the strength/duration (S/D) curve for the modified SENN model employed here for single monopolar and bipolar pulses. The behavior of the model is in agreement with other published studies (Mercadal, 2017; Reilly, 1989; Rogers, W. R. et al., “Strength-duration curve an electrically excitable tissue extended down to near 1 nanosecond,” IEEE Trans. Plasma Sci., vol. 32, no. 4 II, 1587-1599, 2004.).

### Effect of Pulse Width and Delays on Nerve Fiber Response

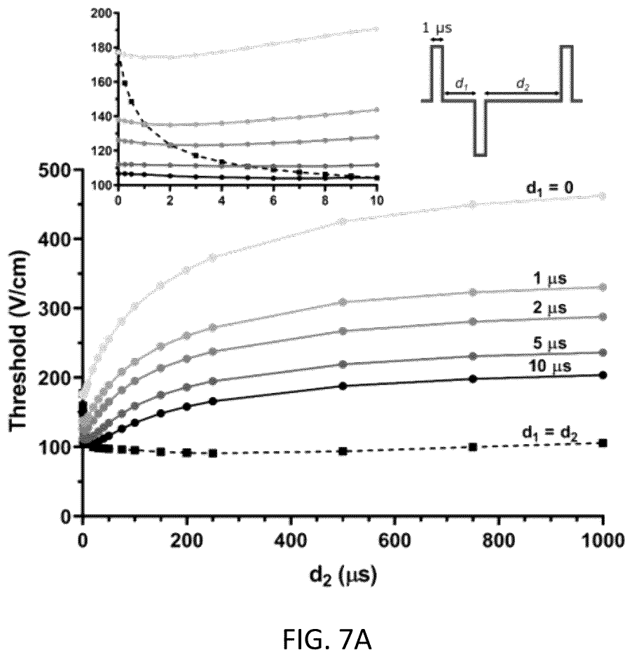

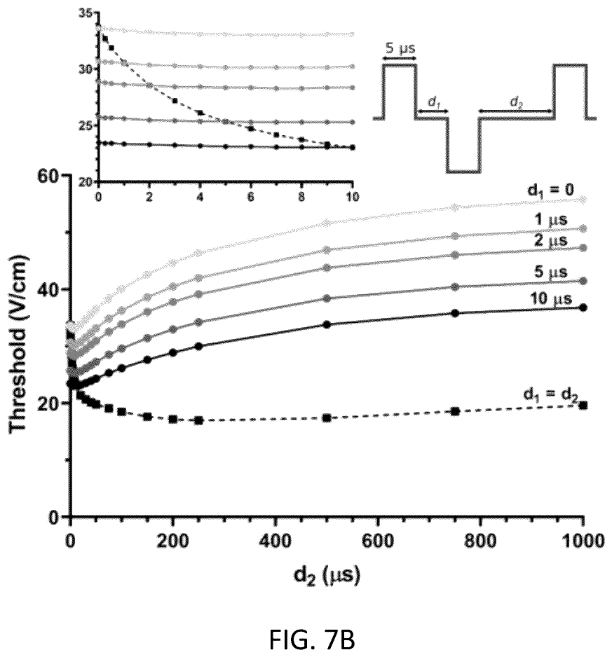

The neural response to varying interphase and interpulse delays is shown in FIGS. 7A-B. Although literature references demonstrate amplified excitability with prolonged interphase gaps for single biphasic pulses, here it is explicitly demonstrated that short interphase delays result in the greatest stimulation thresholds. For symmetric 1 μs pulse bursts, stimulation thresholds decreased 24% and 41% when the interphase delay was increased to 1 or 10 μs from 0. For 5 μs pulse bursts, this effect was not as pronounced, but thresholds fell 9% and 32% across the same delay increases.

For bursts made up of 5 μs pulses, interpulse delays of 1 ms exhibited increased excitation thresholds approximately 60% higher than with no interpulse delay, regardless of the interphase delay. Interestingly, for bursts with 1 μs constitutive pulse widths, holding the interphase delay to a minimum increases the relative gain in stimulation threshold that can be attained while lengthening the interphase delay. For example, a burst comprised of 1 μs pulses with d₁ of 1 μs and d₂ of 1,000 μs exhibits a threshold 2.44× that of a similar burst with d₂ maintained at 1 μs; extending d₁ to 10 μs reduces the gain that can be achieved with this modification in d₂ to 1.95×. Finally, the dashed curve in FIGS. 7A-B indicates that symmetric increases in both delays cause a sharp decline in excitation threshold with a minimum near 250 μs and a gradual recovery with further symmetric increases in d₁/d₂. The trends observed here suggest the optimal strategy for reducing nerve stimulation is to minimize d₁ while maximizing d₂. In light of this observation, subsequent simulations were performed with d₁ fixed at 1 μs to examine the effects of d₂ independently. For practical reasons, d₁ was fixed to 1 μs; most generators used in clinical and pre-clinical studies employ fully-controlled solid-state switches (e.g. MOSFET, IGBT) which have a certain switching time, and when generating bipolar pulses, some time is required during polarity reversal to avoid shorting the DC power supply.

### Effect of Interpulse Delay on Physical Response

To assess the effect of the interpulse delay on ablation, temperature rise, and nerve excitation in a realistic clinical setting, thresholds required to ablate liver cells in a 3D collagen hydrogel were characterized, which informed subsequent construction of a numerical model representative of in vivo treatment. In this model, H-FIRE waveforms with pulse widths of 1 or 5 μs and fixed interphase delay of 1 μs were applied, then the interpulse delay was varied from 0.1 μs to 1,000 μs (Table III). See Example 3 for calculation of upper limit on d₂.

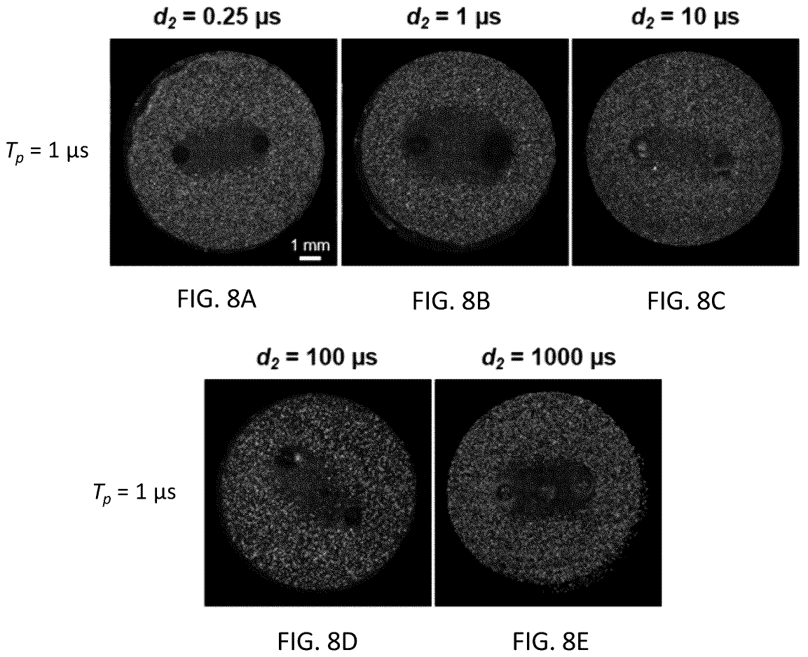

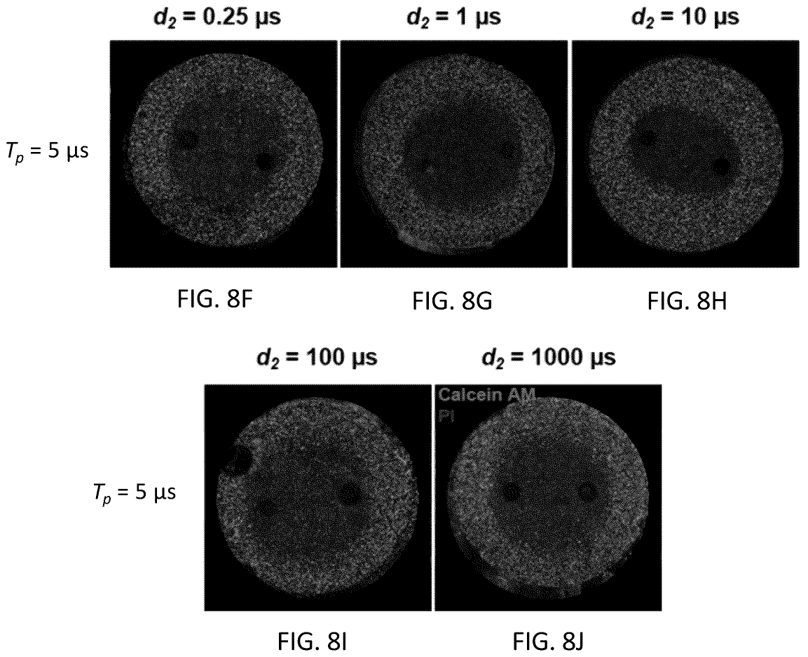

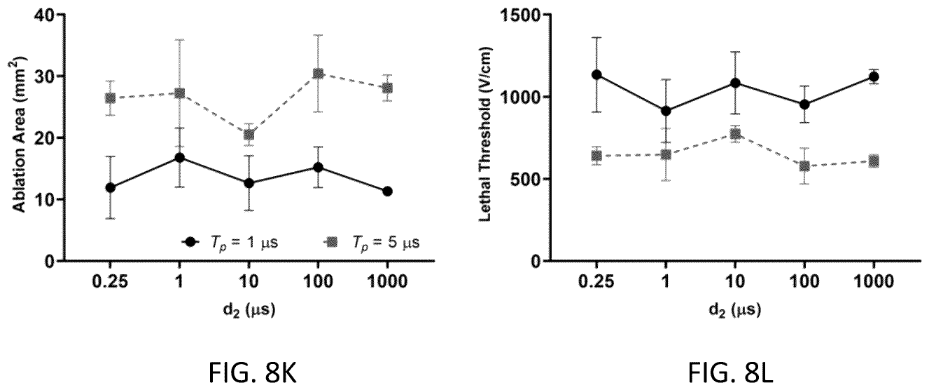

FIGS. 8A-K illustrate the results of cell-laden hydrogel treatment with different variable delay H-FIRE waveforms. Due to generator limitations, the shortest interpulse delay used in experiments was 250 ns. A two-way ANOVA was performed to test the effect of pulse width and interpulse delay, and it was found that only pulse width contributed significantly (p<0.0001) to different lethal EFTs. Due to this result, E₁ was set as the average of all protocols with a given constitutive pulse width, which was 1030±182 V/cm for bursts with 1 μs pulses and 658±116 V/cm for bursts made up of 5 μs pulses. See Example 3 for individual results.

In the model, lengthening the interpulse delay (d₂) reduced the effective baseline conductivity due to the lower characteristic frequency of the burst. This effect is especially prevalent in bursts with constitutive pulse widths of 1 μs, but becomes less significant with pulse widths of 5 μs. As d₂ is extended to 100 μs or longer, the effective baseline conductivity becomes identical regardless of the constitutive pulse width of the burst (Table III). Alternatively, the sigmoid transition range correlates with the relative difference between the reversible EFT and lethal EFT. At or above the lethal EFT, the conductivity of all burst waveforms with a given pulse width converges.

The electric field distribution during the last burst as a function of constitutive pulse width and interpulse delay is shown in FIGS. 9A-D. As the electric field is a function of local conductivity, trends in the field distribution closely follow those of the conductivity as mentioned above. Of note is the increased vertical field exposure but slightly suppressed exposure moving horizontally outward from the electrodes with increased d₂. FIGS. 9E-F demonstrate the percent difference in amount of tissue exposed to varying electric fields compared to a symmetric burst with 1 μs interphase and interpulse delays. These curves were developed by calculating the area of tissue exposed to fields equal to or greater than each field given on the x-axis, then calculating the relative difference between each variable delay curve and the symmetric curve. Areas of exposure to fields of 10 to about 1,500 V/cm are increased with longer interpulse delays. The exception to this trend occurs for a small range of fields near the midpoint of the transition zone in the dynamic conductivity curve for each waveform.

The intersection points between the vertical lines and the curve for each waveform indicate the relative difference in area of reversible (Eᵣₑᵥ) and irreversible (Eₗₑₜₕₐₗ) electroporation compared to a symmetric burst with both delays set to 1 μs. Thus, 1 μs pulse bursts with d₂=1,000 μs give rise to a 17% and 4.9% increase in area of reversible and irreversible electroporation, respectively, while shortening d₂ to 0.1 μs reduces these areas by 2.7% and 0.6%, respectively. Bursts made up of 5 μs pulses exhibit similar trends, with increases in area of 7.6% and 3.2%, respectively, with d₂=1,000 μs versus the symmetric case. By shortening d₂ to 0.1 μs with a 5 μs pulse width, the area of reversible electroporation is reduced by 1% while the irreversibly electroporated region increases 2.3%.

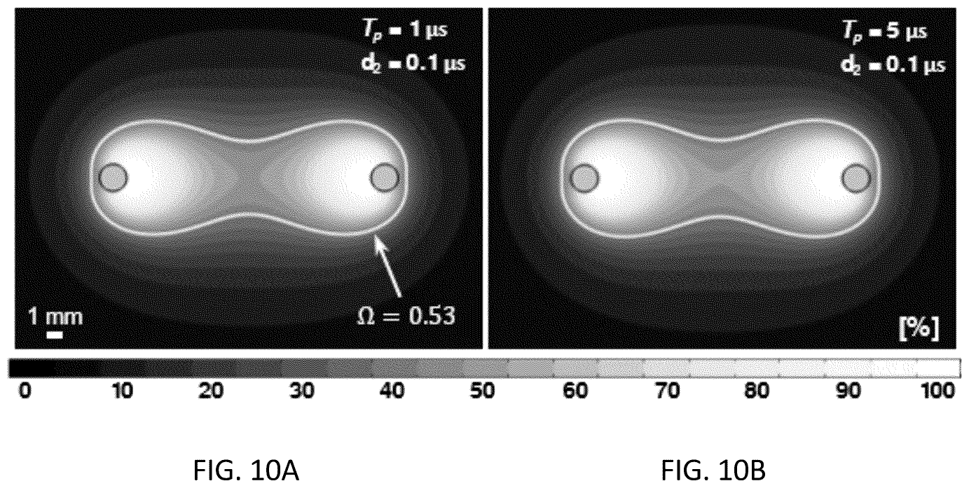

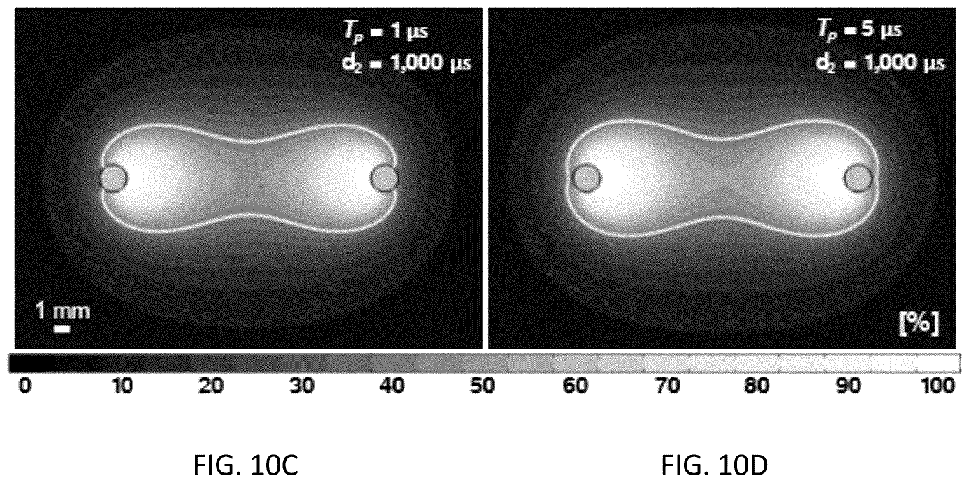

FIGS. 10A-D illustrate the thermal response as a function of modifications in d₂. The figures show the thermal damage distribution arising from treatment with different H-FIRE waveforms expressed as a percentage (P_{Ω}). For 1 μs pulse bursts, the area of thermal damage (Ω>0.53) is reduced from 38.7 mm² to 34.6 mm² as d₂ is extended from 0.1 to 1,000 μs. With the same extension in d₂, thermal damage resulting from treatment with 5 μs pulse bursts is reduced from 40.2 mm² to 39.3 mm². Additionally, the maximum value of 22 is inversely related to d₂. Bursts with constitutive pulse width of 1 μs exhibit maximum values of 5.7 and 4.1 for d₂=0.1 and 1,000 μs. Applying the same changes in d₂, 5 μs burst waveforms exhibit maxima of 6.1 and 5.8, respectively. This can be attributed to higher temperatures adjacent to the electrodes with shorter delays due to the increased electrical conductivity.

### Effect of Interpulse Delay on Relative Efficacy

Next, a metric to define the relative efficacy of a given waveform is introduced:

$${R}_{eff} = \frac{{A}_{IRE}}{{A}_{TD}} \frac{{A}_{IRE}}{{A}_{EXC}} = \frac{{A}_{IRE}^{2}}{{A}_{TD} \cdot {A}_{EXC}} \qquad ( Equation 1 )$$

where R_{eff} is a dimensionless quantity representative of the efficacy of the waveform and A_{IRE}, A_{TD}, and A_{EXC} are the areas of irreversible electroporation, thermal damage, and excitation. This value can be used to compare the ability of each waveform to create ablations while limiting the extent of thermal damage and nerve excitation.

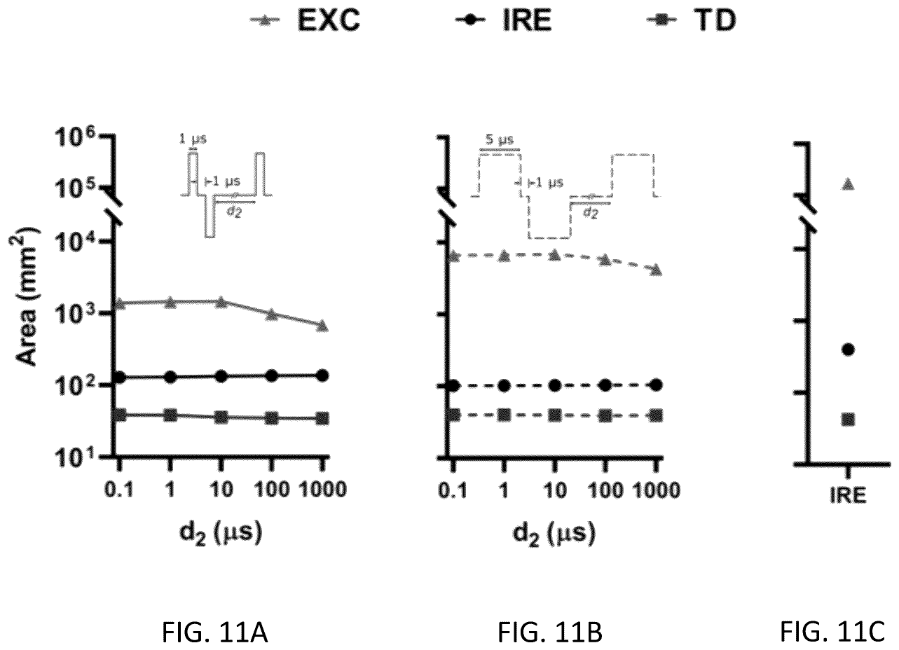

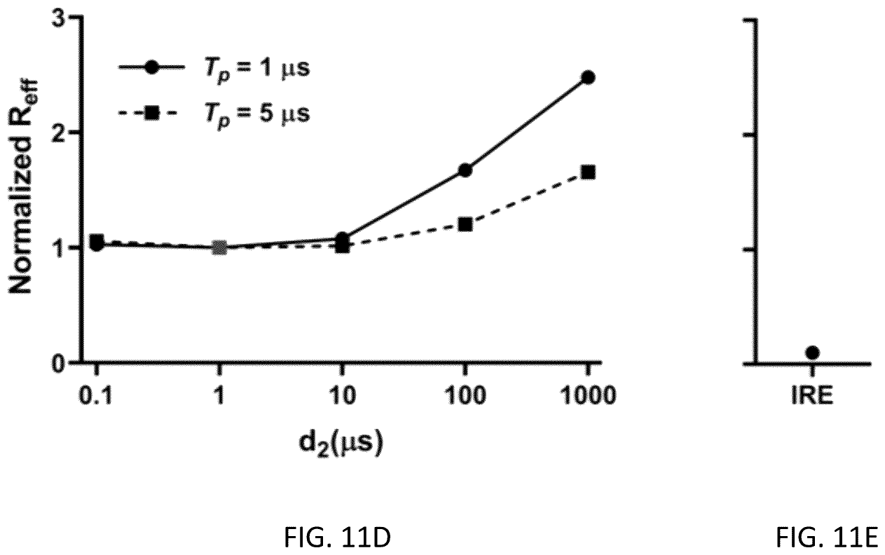

Calculated values of A_{IRE}, A_{TD}, and A_{EXC} are shown in FIGS. 11A-C. A minor increase in ablation area is observed with longer interpulse delays, while areas of excitation and thermal damage are reduced. Trends in R_{eff} versus d₂ are shown in FIGS. 11D-E. For visualization, R_{eff} for bursts with either constitutive pulse width was normalized to the case in which d₂=1 μs. Here, it can be seen that R_{eff} increases exponentially with d₂ on a log scale. This increase is continuous for 1 μs pulse bursts, but is not obvious until d₂ reaches 100 μs for bursts with constitutive pulse widths of 5 μs. For 1 μs pulse bursts, increasing d₂ from 0.1 μs to 1,000 μs increased R_{eff} nearly 3-fold from 0.35 to 0.84. Similar modifications in d₂ for 5 μs pulse bursts increased R_{eff} from 0.23 to 0.36. For comparison, R_{eff} for the modeled conventional IRE protocol was 0.022, which was mainly dominated by the large area of nerve stimulation.

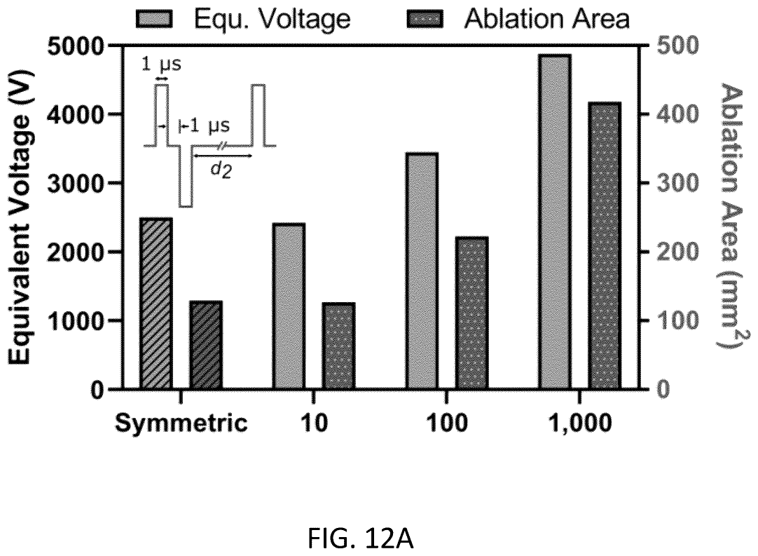

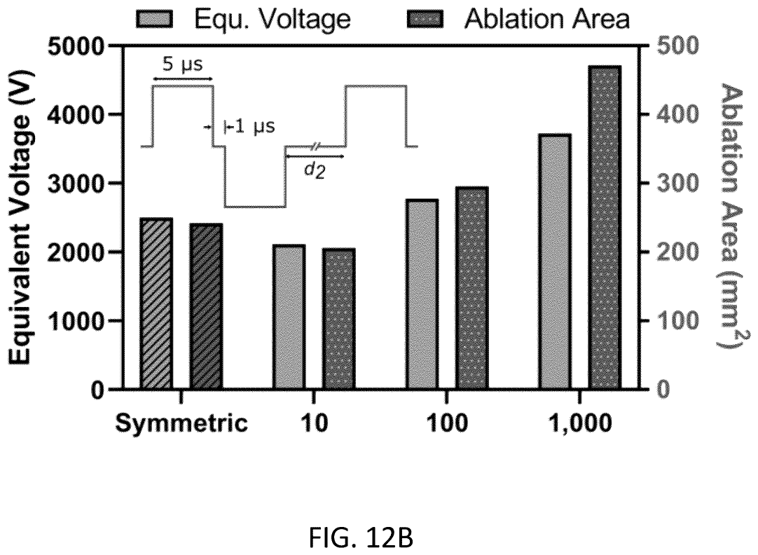

Finally, a parametric analysis was performed for each variable delay waveform with extended d₂ to determine the maximum voltage that can be applied while maintaining the same excitation area as the case in which d₁/d₂ are symmetric and equal to 1 μs (FIGS. 12A-B). For symmetric waveforms comprised of 1 μs and 5 μs pulse bursts, the standard treatment with 100 bursts at 2,500 V produced ablations of 130 mm² and 236 mm², respectively, and stimulation areas of 1,298 mm² and 6,341 mm². For 1 μs burst waveforms, extending d₂ to 10 μs, 100 μs, or 1,000 μs gave maximum voltages that could be applied of 2,422 V, 3,450 V, and 4,878 V, respectively, while maintaining a stimulation area of 1,298 mm². At these voltages, ablation areas are 127 mm², 123 mm², and 418 mm², respectively. Waveforms with 5 μs constitutive pulses exhibit maximum voltages of 2, 116 V, 2,777 V, and 3,724 V when d₂ is 10 μs, 100 μs, or 1,000 μs, respectively. Applying bursts at these voltages gives ablation areas of 206 mm², 296 mm², and 472 mm², while maintaining a stimulation area of 6,341 mm².

Variable delays within H-FIRE bursts may be more clinically efficacious in terms of ablating tissue with reduced nerve excitation in comparison to current waveforms with symmetric delays. To evaluate the feasibility of such an approach, the effects of variable delays on neural excitation were analyzed for bursts with constitutive pulse widths of 1 μs and 5 μs. Based on these findings, the interphase delay was fixed to 1 μs and a parametric sweep of the interpulse delay in a realistic two-needle treatment model was performed to quantify the extent of ablation, excitation, and thermal damage.

The results suggest that the largest clinical effect will be reduced nerve excitation with extended interpulse delays. For waveforms with short constitutive pulse widths near 1 μs, interpulse delay has a substantial influence on the characteristic frequency of the burst, and thus the effective baseline conductivity and Joule heating produced from treatment. This difference in baseline conductivity as a function of d₂ is dependent upon the slope of the tissue's impedance spectrum across the range of frequencies of interest. Thus, the effects of modifying the interpulse delay on electrical conductivity and Joule heating may vary between tissues.

As shown in FIGS. 7A-B, prolonging d₂ yields a marked increase in the electric field required to stimulate a nerve fiber compared to a burst with short, symmetric delays. However, it should be noted that with longer burst durations, it is possible that a single burst will initiate multiple action potentials in excitable cells exposed to threshold stimuli or greater. This is due to the fact that action potentials—and, in turn, refractory periods—in skeletal muscle and nerve are roughly 2 to 4 ms in duration. In the case that extremely long values of d₂ (250-1,000 μs) are not clinically efficacious, shorter delays that maintain a burst duration just below the absolute refractory period of muscle will likely still be desirable. For example, for a 5 μs pulse burst with 100 μs of energized time and d₁ set to 1 μs, d₂ can be extended to 146 μs assuming an absolute refractory period of 3 ms for muscle (see Example 3 for calculation). By maintaining a burst duration that can be delivered while the muscle is refractory, a single action potential would be generated in affected cells, mitigating synergistic effects such as frequency summation that might occur due to multiple action potentials firing in response to each burst. This approach has been taken for ECT protocols (8×100 μs pulses) and in an in vivo rat study, it was found that the number of contractions could be reduced from eight lighter contractions to a single stronger contraction by increasing the repetition rate to 100 Hz or more (Miklavčič, D. et al., “The effect of high frequency electric pulses on muscle contractions and antitumor efficiency in vivo for a potential use in clinical electrochemotherapy,” Bioelectrochemistry, vol. 65, 121-128, 2004.). While further increases in frequency reduced the strength of the single contraction, this finding suggests that bursts with durations in the range of 70 ms will be feasible, but this relationship must be more rigorously explored to definitively place limits on d₂.

It is important to appreciate the mechanism by which prolonged values of d₂ suppress nerve fiber activation. Because these fibers are much longer than most cells in the tissue parenchyma, membrane charging occurs via a unique mechanism, relying on charge redistribution along the length of the fiber rather than the potential difference between the interior and exterior of the fiber at a given point. As a result, while parenchymal cells exhibit charging constants on the order of 1 μs, membrane charging constants of nerve fibers are much longer, and are typically reported to be on the order of hundreds of microseconds. Thus, extending d₂ to tens or hundreds of microseconds allows the nerve fiber membrane to discharge between subsequent bipolar pulses within the burst.

Excitation is also dependent on d₁, which must be shortened sufficiently for the negative phase of each bipolar pulse to adequately cancel VGSC activation by its corresponding positive phase. Conversely, interphase delays on the order of the time constant of targeted cells increase time of exposure to a critical transmembrane potential, which has been shown to correlate with permeabilization. This implies an optimal value of d₁ exists that mitigates VGSC activation while also allowing passive discharge of targeted cellular membranes. In this study, d₁ was fixed to 1 μs (˜τₘ) to demonstrate theoretically that these mechanisms can be exploited to apply higher potentials without increased stimulation, which will allow for larger areas of ablation and permeabilization. To achieve desirable results without substantial temperature increases, thermal mitigation strategies can be implemented, such as the use of phase change electrodes and/or by internal electrode cooling.

In this in vitro study, it was found that lethal EFTs were only a function of the width of constitutive pulses. Bursts with 1 μs pulse widths exhibited a threshold near 1 kV/cm, approximately 57% higher than those with 5 μs widths, indicative of the bipolar cancellation present with very short pulses. Recent experimental data have found that interphase and interpulse delays mediate permeabilization and lethality of high-frequency pulse bursts, with longer delays typically magnifying biological effects. For instance, Valdez and colleagues found that bipolar nanosecond pulses with interphase delays greater than 10 ms resulted in permeabilization comparable to that of energy-matched monopolar pulses (Valdez, C. M. et al., “The interphase interval within a bipolar nanosecond electric pulse modulates bipolar cancellation,” Bioelectromagnetics, vol. 39, no. 6, 441-450, 2018). And Polajzer and colleagues demonstrated that assisted discharge can explain cell viability trends resulting from symmetric increases in interphase and interpulse delay, but specific cases of cell permeabilization deviated from theoretical projections (Polajžer, T. et al., “Cancellation effect is present in high-frequency reversible and irreversible electroporation,” Bioelectrochemistry, vol. 132, 2020). Recently, increased cell death has also been achieved by independently lengthening the interpulse delay up to 1 ms for a single burst with 800 μs of energized time (Vižintin, A. et al., “Effect of interphase and interpulse delay in high-frequency irreversible electroporation pulses on cell survival, membrane permeabilization and electrode material release,” Bioelectrochemistry, vol. 134, August 2020). Thus, while the present inventors did not find a significant impact of d₂ on lethal thresholds, it is possible that in other cell types or in bulk tissue, extended values of d₂ may exhibit lower thresholds than the symmetric bursts currently being used. Importantly, if this is realized clinically, R_{eff} will be further amplified as d₂ is prolonged.

By assuming a constant perfusion rate, simulations neglect to account for local disruptions in blood flow, and may underestimate temperature rise. The present inventors assumed electrical properties of tissue exposed to sub-electroporative H-FIRE bursts could be defined by determining a discrete characteristic frequency at which the burst operates. Next, it was assumed that this characteristic operating frequency f_{c} is defined as the inverse of the bipolar pulse period (FIGS. 1B-C). This assumption has been employed previously, but not for high-frequency bipolar waveforms with variable delays as introduced here (Zhao, Y. et al., “Characterization of conductivity changes during high-frequency irreversible electroporation for treatment planning,” IEEE Trans. Biomed. Eng., vol. 65, no. 8, 1810-1819, 2018). Thus, it remains to be determined whether extensions in d₂ continuously generate reductions in f_{c}, or whether there is a limit to the influence of d₂ on f_{c}. Additionally, σ_{f} was calculated by assuming the β-dispersion of liver tissue plateaus at 10 MHz. So long as each waveform saturates to the same conductivity, lengthening d₂ will produce the benefits reported in this invention regardless of the value of σ_{f}.

It should be noted that the nerve excitation results are independent of the assumptions underlying the conductivity curves constructed, and in their own right provide valuable information toward selecting delays within H-FIRE waveforms. However, it is also important to recognize that the nerve stimulation model has limitations. To facilitate translation to the 2D ablation models, thresholds for excitation were characterized at the terminus of a 6-node fiber. This truncated fiber model could introduce inaccuracies in terms of the current distribution that would not be present in a longer fiber. Additionally, the theoretical time constant of the model is high (τₑ=626 μs) compared to the original SENN model (τₑ=120 μs) exposed to uniform-field excitation, which reflects the small diameter and short length of the fiber modeled here and falls within the range of experimental values for mammalian nerve. Finally, areas given for excitation are areas in which a nerve terminus could be excited if aligned with a relatively constant field at threshold or higher. While these areas build intuition and facilitate comparison, they are not precisely indicative of the areas of tissue that will be stimulated, but surrogates that represent the relative magnitude of excitation (and thus, contraction).

Irreversible electroporation is an emerging focal treatment modality for solid tumors and in cardiac ablation for treating atrial fibrillation. While promising results have been reported, adoption of IRE has been hindered by its complex anesthetic regimen and treatment protocols. H-FIRE has been introduced to overcome these limitations and drastically simplifies clinical procedures, but waveforms suffer from reduced ablation volumes compared to their IRE counterparts. For this reason, higher voltages are often desired during H-FIRE, but this increases the likelihood of thermal damage and muscle contractions. Here, it is demonstrated that minor changes to the delays within H-FIRE burst waveforms may suppress neural excitation and Joule heating. It is also possible that these waveforms will increase ablation size by modulating local electrical conductivity. These modified waveforms be readily implemented without generator hardware modifications or systemic changes to existing treatment protocols.

### Example 3

### Determining Maximum Interpulse Delay

To determine the range of reasonable values of d₂, the fact that current H-FIRE protocols consist of 100-300 bursts delivered at a repetition rate of up to 1.5 Hz was considered. To maximize clinical relevance, the burst duration should be amenable to incorporation into protocols in which ECG synchronization is desired and/or necessary. Considering that the absolute refractory period of cardiac myocytes is 250-300 ms, an upper limit of 100 ms was placed on the burst duration. This gives ample time for the R-wave to be detected and pulse delivery to be initiated. To find the maximum value of d₂ that meets this constraint, the following equation giving the burst duration Ty was used:

$${T}_{b} = N \cdot ( 2 {T}_{p} + {d}_{1} + {d}_{2} ) - {d}_{2} \qquad ( Equation 2 )$$

In Equation 2, N is the number of bipolar pulses within the burst-chosen such that 2N·Tₚ=100 μs. Waveforms with constitutive pulse widths of 1 μs require that N=50, while 5 μs pulse bursts require N=10. Thus, with the same delay structure, burst duration is inversely related to constitutive pulse width, so the maximum value of d₂ was determined by substituting 100 ms for T_{b}, 50 for N, and 1 μs for d₁ in (S1), then solving for d₂. This gives a maximum interpulse delay of 2.04 ms, which was lowered to the nearest power of 10 for integration into the parametric analysis. It is worth noting that d₂ can be prolonged up to 11.1 ms while meeting the above constraints for bursts with constitutive pulse widths of 5 μs.

### Modified SENN Model Current Formulations

Ionic currents across the nerve fiber membrane were calculated according to the Hodgkin-Huxley formulations, with gating parameters and conductances empirically determined for mammalian nerve fibers at 36° C.

### Experimental Determination of Lethal Electric Field Thresholds

An established technique was used to characterize thresholds required to electroporate and ablate malignant liver cells in response to various waveforms. Below the experimental approach is summarized.

### 3D Collagen Hydrogel Fabrication

Human hepatocellular carcinoma cell line Hep G2 [HEPG2] (ATCC® HB-8065™) was cultured according to supplier recommendations and passaged regularly at 70-80% confluence. Hydrogels were fabricated by mixing high concentration collagen I from rat tail tendons with 10× culture media (10% v/v) and 1N NaOH (2% collagen volume), and stirring until homogenous with a spatula. NaOH was added to adjust the pH to 7.2-7.4 (confirmed visually), and cells in suspension were added to achieve a final collagen density of 5 mg/mL and cell concentration of 10⁶ cells/mL. After homogenizing, the solution was injected into custom polydimethylsiloxane (PDMS) inserts—each in one well of a 24-well culture plate—such that collagen was molded into disks—each with a radius of 5 mm and thickness of 1 mm. The disks were allowed to polymerize for 20 minutes at 37° C., covered in fresh media, and returned to the incubator overnight prior to treatment.

### Pulsed Electric Field Treatment Delivery

Approximately 24 hours after seeding, cells were transferred to a mobile incubator housed under sterile conditions to maintain physiological temperature of 37° C. Prior to treatment, media was aspirated and a custom holder was used to insert two stainless steel electrodes (Ø=0.9 mm) with center-to-center separation of 4 mm within the gels. H-FIRE burst waveforms were generated by a custom bipolar pulse generator (EPULSUS FBM1-5®, Energy Pulse Systems, Lisbon, Portugal) while IRE pulses were delivered with a square wave electroporation system (ECM 830, BTX, Holliston, MA). In the case of H-FIRE, the total number of bipolar pulses was adjusted to achieve an energized time of 100 μs across all waveforms, and 100 bursts were delivered at a rate of 1 burst/second. For IRE, 100 monopolar pulses with Tₚ=100 μs were delivered at a rate of 1 pulse/second. In all cases, 600 V was applied across the electrodes; upon completion of treatment, hydrogels were covered in fresh media and returned to incubation at 37° C. and 5% CO₂.

### Measurement of Lesion Areas and Calculation of Electric Field Thresholds

To characterize ablation sizes, cells were incubated at 37° C. and 5% CO₂ for 24 hrs after treatment, then stained with a solution of 2.5 μM calcein AM (green, live cells) and 22 μM propidium iodide (red, dead cells) in PBS. After incubating for 30 minutes, hydrogels were washed twice with PBS and imaged using an inverted confocal microscope (Observer Z1, Carl Zeiss AG, Oberkochen, Germany). Ablated areas were then measured manually in ImageJ (NIH, Bethesda, MD). For reversible tests, the above staining and imaging procedure was performed immediately after treatment. A numerical model (Comsol Multiphysics 5.5, COMSOL, Stockholm, Sweden) of the hydrogel treatments was constructed, and the field distribution was computed. An exponential curve relating areas of exposure to different electric field magnitudes was constructed, and measured areas were fed into this curve to compute corresponding minimum thresholds required to electroporate or ablate this area. The results of this analysis are summarized in Table IV.

TABLE IV
Lethal EFTs for Variable Delay H-FIRE Burst Waveforms

| Tₚ (μs) | d₁ (μs) | d₂ (μs) | Areas (cm²) | EFT (V/cm) | n |
|---|---|---|---|---|---|
| 1 | 1 | 0.25 | 11.9 ± 5.0 | 1134 ± 227 | 6 |
| 1 | 1 | 1 | 16.8 ± 4.8 | 913 ± 191 | 7 |
| 1 | 1 | 10 | 12.6 ± 4.4 | 1083 ± 189 | 5 |
| 1 | 1 | 100 | 15.2 ± 3.3 | 953 ± 111 | 7 |
| 1 | 1 | 1,000 | 11.3 ± 0.8 | 1122 ± 44 | 5 |
| 5 | 1 | 0.25 | 26.4 ± 2.8 | 641 ± 56 | 5 |
| 5 | 1 | 1 | 27.2 ± 8.7 | 647 ± 158 | 6 |
| 5 | 1 | 10 | 20.5 ± 1.8 | 774 ± 51 | 6 |
| 5 | 1 | 100 | 30.4 ± 6.2 | 577 ± 109 | 5 |
| 5 | 1 | 1,000 | 28.1 ± 2.1 | 608 ± 38 | 3 |
| 100 | — | — | 40.6 ± 4.1 | 429 ± 47 | 4 |
| Reversible Test | | | | | |
| 5 | 1 | 1 | 38.4 ± 0.8 | 4539.4 | 3 |

### Statistical Analysis

A two-factor analysis of variance (ANOVA) was performed to test whether pulse width (Tₚ) or interpulse delay (d₂) had an effect on the computed lethal EFTs. Where appropriate, a post-hoc Tukey's HSD test was used for pair-wise comparisons. All statistical analyses were performed using Prism (version 8.3.1, GraphPad Software, San Diego, CA) with α of 0.01.

### Example 4

Methods described herein allow a user to modify the temporal nature (FIGS. 1A-C) of bursts of bipolar and/or monopolar pulsed electric fields in order to maximize therapeutic outcomes while reducing side effects. FIGS. 1A-C provide representative idealized voltage waveforms of an IRE pulse (FIG. 1A), symmetric H-FIRE burst (FIG. 1B), and asymmetric H-FIRE burst (FIG. 1C). Each IRE pulse is characterized by an amplitude (1) and pulse width (2), and is repeated at a specific interval (3). Symmetric HFIRE bursts (B) are comprised of short bipolar pulses—each with a positive pulse of amplitude (1) and width (2), an intraphase delay (4), and an identical negative pulse (i.e. \textcircled{6}=\textcircled{2}+\textcircled{4}+\textcircled{2}+\textcircled{5}. Each bipolar pulse is separated by an interpulse delay (5) equal to (4), and is repeated N times to achieve a desired “energized time.” Asymmetric H-FIRE bursts (C) are similar to (B) with the unique difference that (4)≠(5).

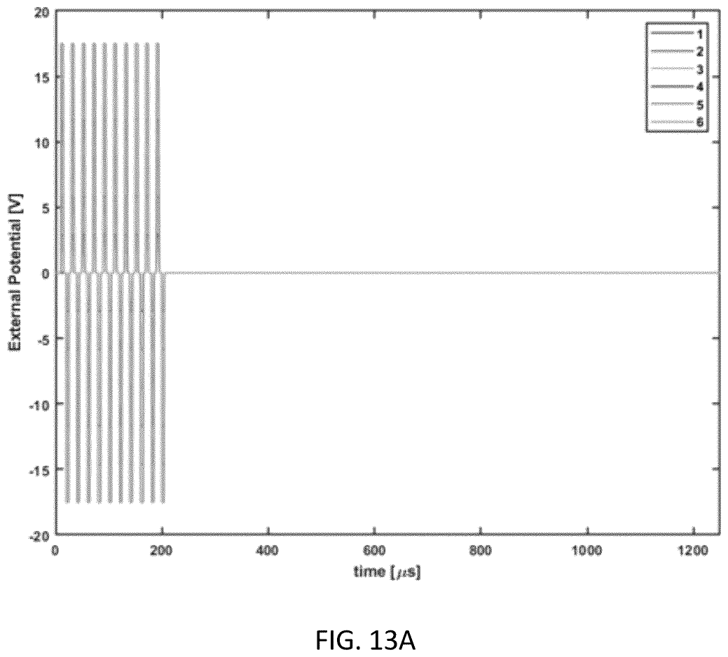

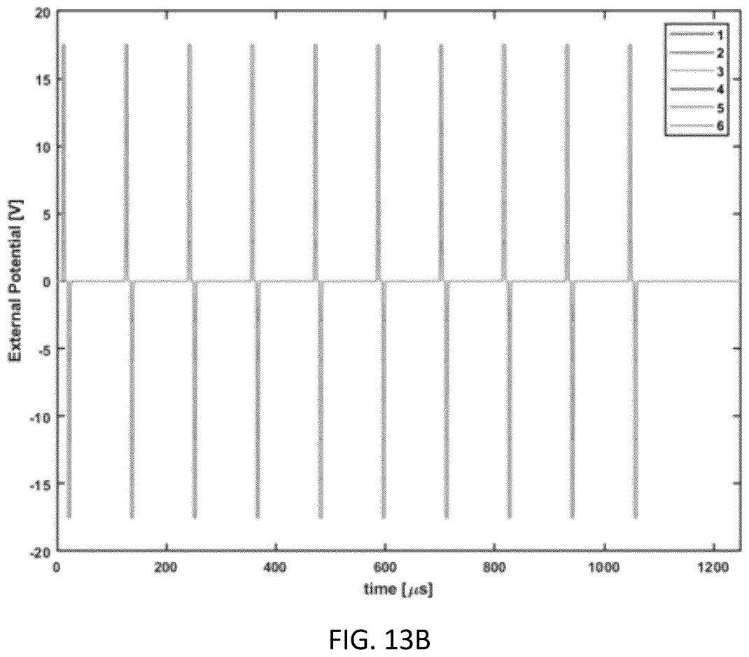

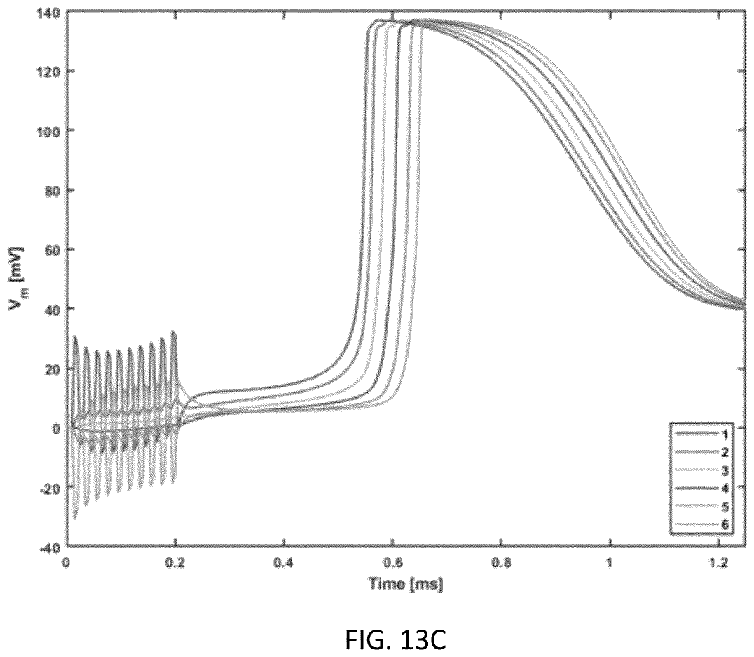

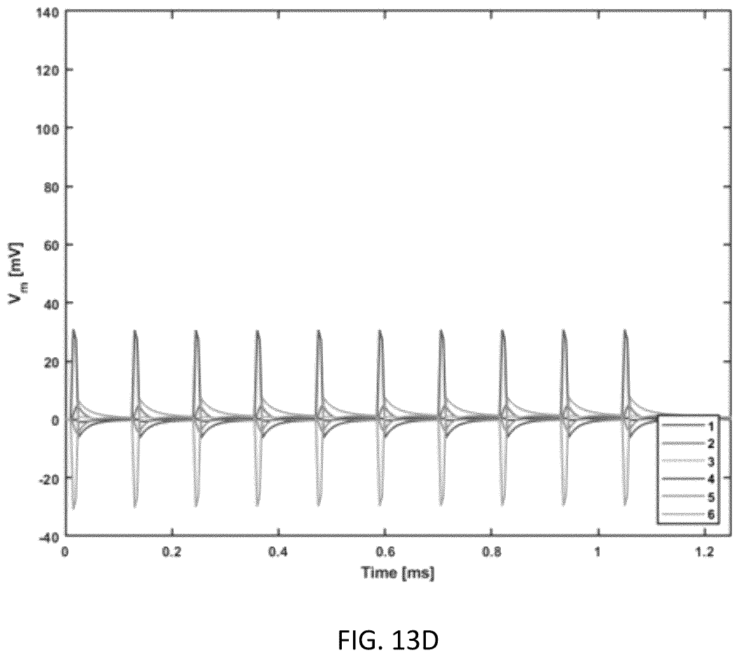

Namely, it is shown mathematically that extensions in the delay between bipolar pulses within the burst reduce the likelihood that the pulses will excite nerve fibers (FIGS. 13A-D). In order to adequately bound the range of pulse widths that this method may be useful for, the threshold field required for nerve fiber excitation when exposed to single bipolar and unipolar pulses is described (FIG. 6).

FIGS. 13A-D provide representative externally applied electric fields and resulting nerve fiber responses. External potential at each of the 6 nodes in the model arising from bursts with 5 μs constitutive pulse widths with d₁ set to 5 μs and d₂ set to 5 μs (FIG. 13A) and 100 μs (FIG. 13B). Normalized membrane potentials are given to the above waveforms in FIGS. 13C-D. Applied electric field was 25.4 V/cm in both cases.

FIG. 6 provides a baseline estimate of the maximum pulse widths for which employing a bipolar regime is efficacious. As seen in this figure, with pulse widths beyond roughly 100 μs, little benefit is gained by applying an additional pulse of alternate polarity. Because bipolar pulses in the microsecond range tend to have reduced biological outcomes versus their monopolar counterparts, the main attraction of bipolar pulses is their reduced stimulation of excitable cells. Thus, pulses of single polarity longer than ˜100 μs likely will not be clinically pursued. It is possible that bipolar pulses with even shorter pulse widths than this would cause stimulation of sufficient magnitude that neuromuscular blocking agents would be required to manage contractions. Thus, it is unknown what upper limit of Tₚ will be clinically realized, but it is important to point out from a theoretical stance that employing a bipolar approach for Tₚ>100 μs seems futile from an excitation standpoint. However, bipolar pulse waveforms may still be desired due to their reduced release of metallic contaminants; further, for very long pulse widths up to 500 μs in length, bipolar approaches may have other advantages such as more uniform permeabilization, but there is no advantage from an excitation perspective.

While current burst waveforms used with therapeutic electroporation-based endpoints provide a number of benefits over traditional IRE, here a relatively rigid framework is described which can be used to more systematically construct these burst paradigms and potentially optimize outcomes.

A constraint dictating the temporal nature of the burst waveform is the cardiac absolute refractory period. Electrical pulses with potential to induce cardiac asynchrony are delivered within this window of roughly 250-300 ms during which cardiac myocytes are refractory, regardless of stimulus amplitude. Thus, for protocol planning purposes, an upper bound which allows for R-wave detection and pulse initiation and completion within this timeframe should be selected. Depending on generator capabilities, R-wave detection software and triggering devices, this upper bound may be extended to 200 ms or more, but for the sake of demonstration, a bound of 100 ms is chosen, which should provide substantial safety margins.

The following equation describes the duration T_{b} of a burst of bipolar pulses:

$${T}_{b} = N \cdot ( {T}_{p 1} + {T}_{p 2} + {d}_{1} + {d}_{2} ) - {d}_{2} \qquad ( Equation 3 )$$

where N is the number of bipolar pulses in the burst, Tₚ₁ and Tₚ₂ are the duration of each pulse of positive and negative polarity, respectively, and d₁ and d₂ are the intraphase and interpulse delay, respectively. Given that Tₚ₁ and Tₚ₂ are conventionally equivalent, and that N is usually chosen based on a total energized time Tₑ (N=Tₑ/2Tₚ), equation 3 can be rewritten as:

$${T}_{b} = {T}_{e} [ 1 + \frac{{d}_{1} + {d}_{2}}{2 {T}_{p}} ] - {d}_{2} \qquad ( Equation 4 )$$

where Tₚ describes the duration of pulses of either polarity. In most protocols, Tₑ is chosen to be 60-200 μs, and Tₚ is between 250 ns and 50 μs. Additionally, d₁ is typically 1-10 μs in duration.

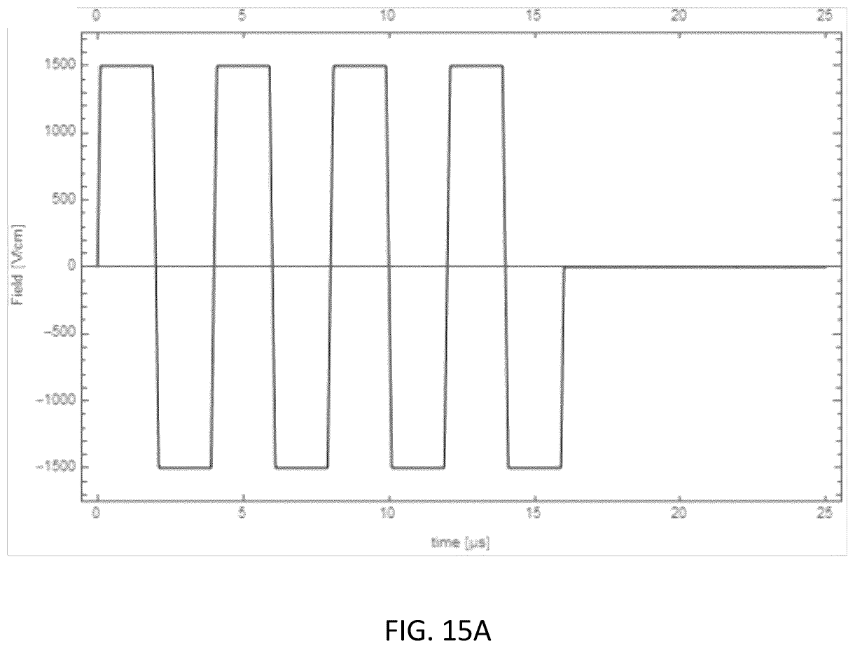

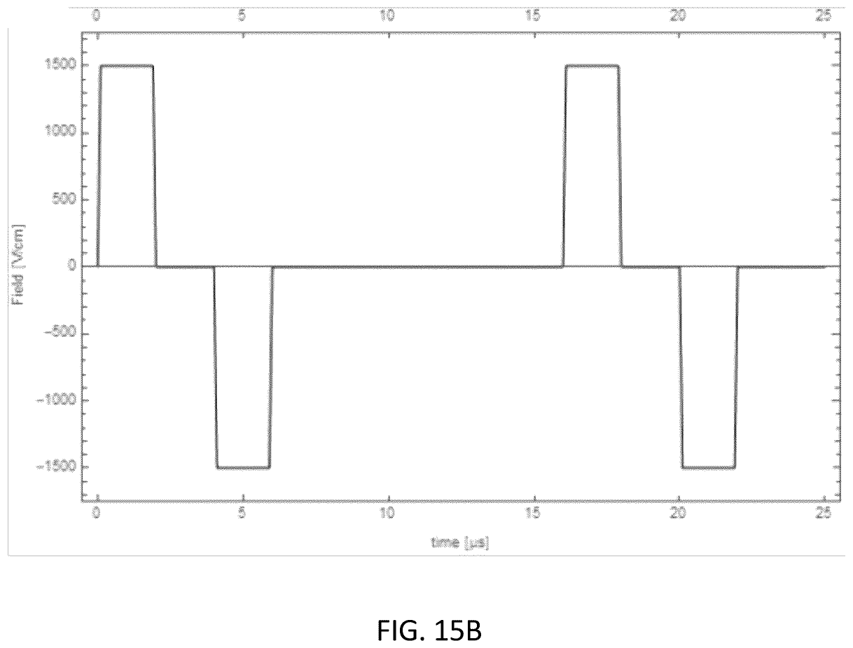

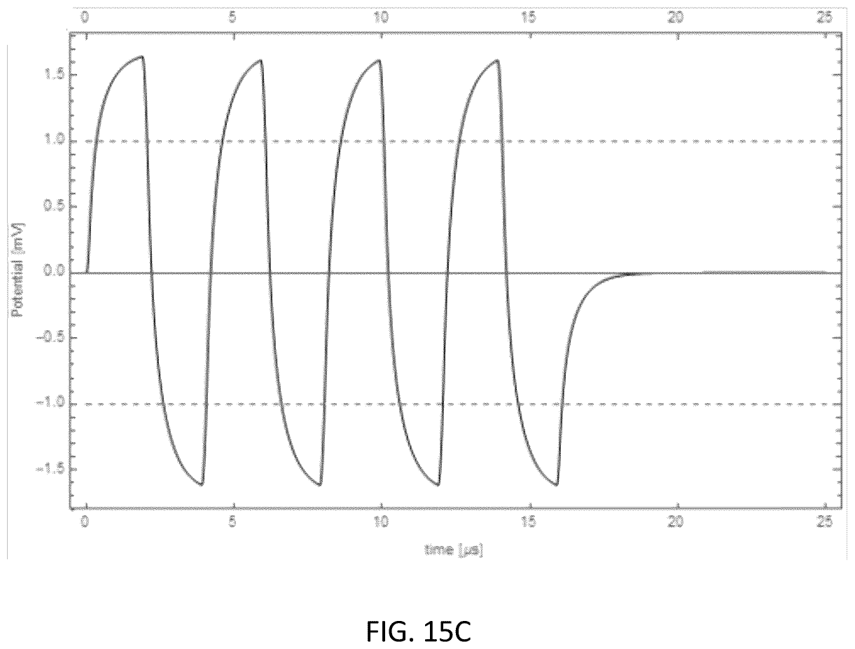

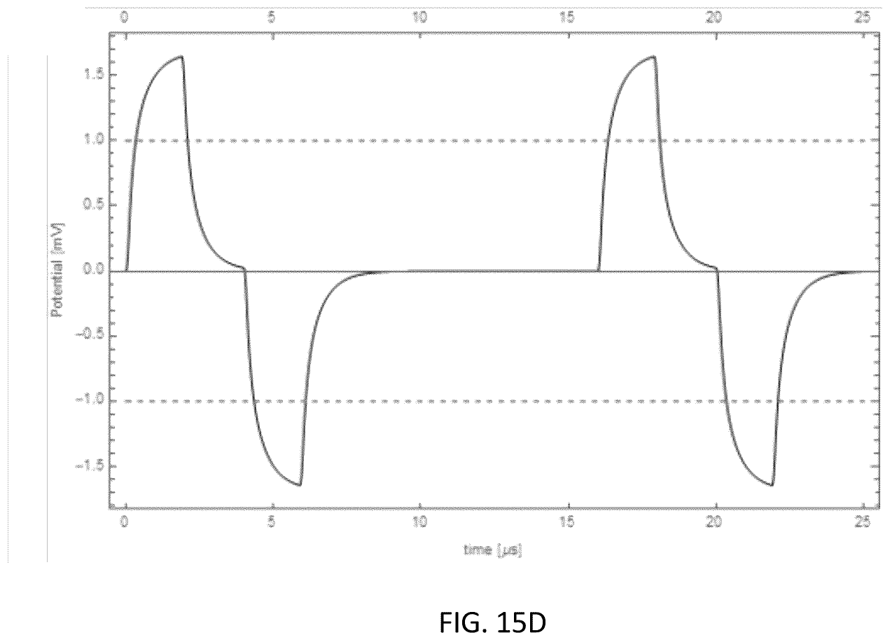

FIGS. 15A-D show the effect of incorporating delays on the induced transmembrane potential experienced by targeted cells. From this graph, it is obvious that introducing an intraphase delay increases the time the cell is above the critical threshold of 1 V. Moreover, by prolonging the interpulse delay up to 10 μs, assisted discharge is completely avoided, maximizing both the duration which the transmembrane potential is non-zero as well as the time the cell is exposed to the critical threshold.

Non-zero values of d₁ allow the transmembrane potential to remain above a critical value of 1 V for a longer duration (FIGS. 15A-D), and put less stress on MOSFETs. However, d₁ should be maintained at the shortest reasonable value that provides these benefits, as extensions in d₁ reduce the maximum achievable excitation threshold (FIGS. 7A-B).

FIGS. 7A-B demonstrate the effect of modifying intraphase and interpulse delays on the excitation thresholds for a nerve fiber. It is apparent here that increases in d₁ are unfavorable from an excitation standpoint, but extensions of d₂ asymptotically increase the threshold toward the value of a single bipolar pulse with given constitutive pulse width and intraphase delay.

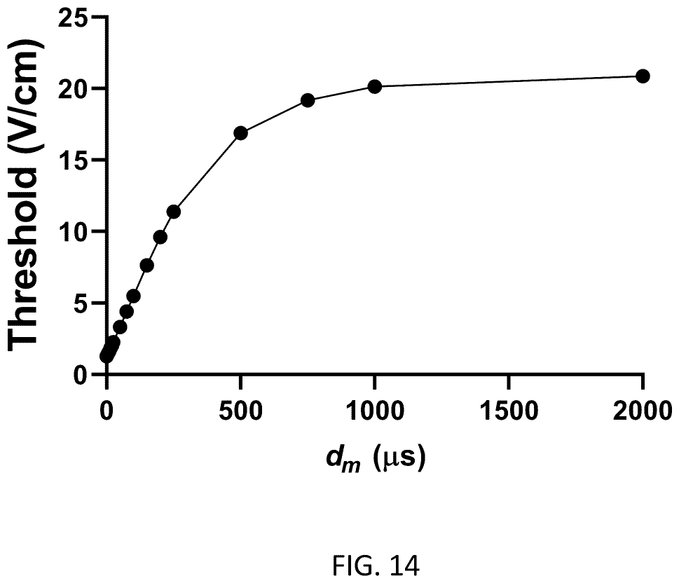

In the given examples in FIG. 14, d₁ was either maintained as equivalent to d₂ or fixed at 1 μs. FIG. 14 illustrates that prolonging dm increases excitation threshold for bursts of monopolar pulses with 1 and 5 μs constitutive pulse widths.

With d₁ chosen, equation 4 can be rearranged to solve for the maximum value of d₂:

$${d}_{2} = \frac{{τ}_{b} - {T}_{e} - N \cdot {d}_{1}}{N - 1} \qquad ( Equation 5 )$$

Bursts of monopolar pulses are characterized by a single delay dm, which can be calculated by:

$${d}_{m} = \frac{{T}_{p} ( {T}_{m} - {T}_{e} )}{{T}_{e} - {T}_{p}} \qquad ( Equation 6 )$$

where Tₘ is the monopolar burst duration. In equations 5 and 6, the maximum period of the burst is substituted for T_{b}/Tₘ (100 ms in the examples). Depending on the application, this value may be the cardiac refractory period, skeletal myocyte refractory period, or any other time window in which the total burst should be delivered.

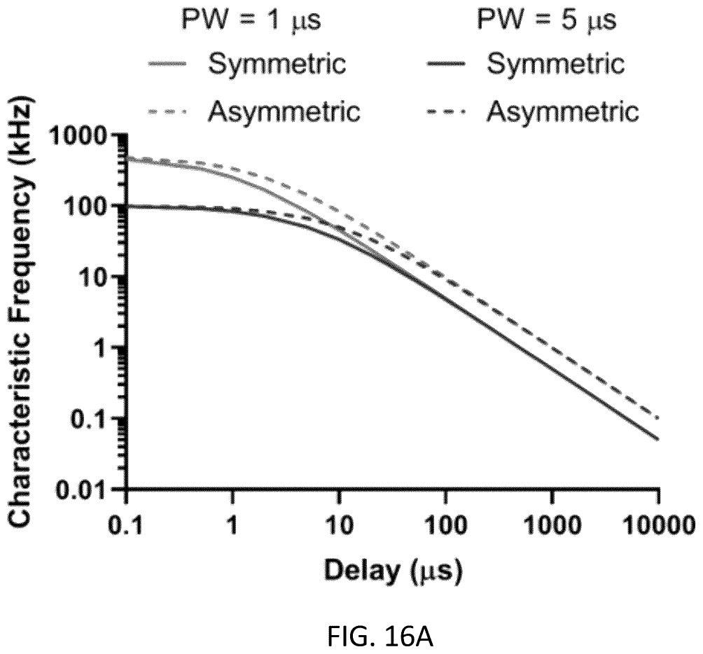

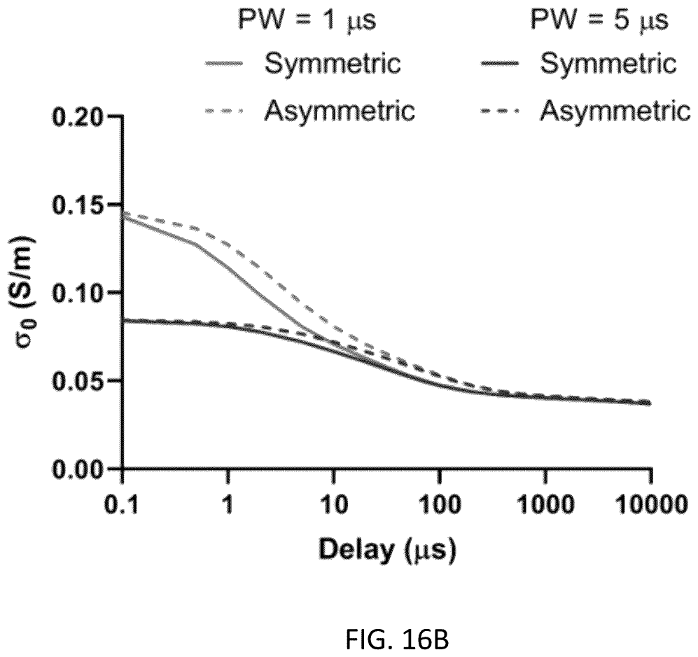

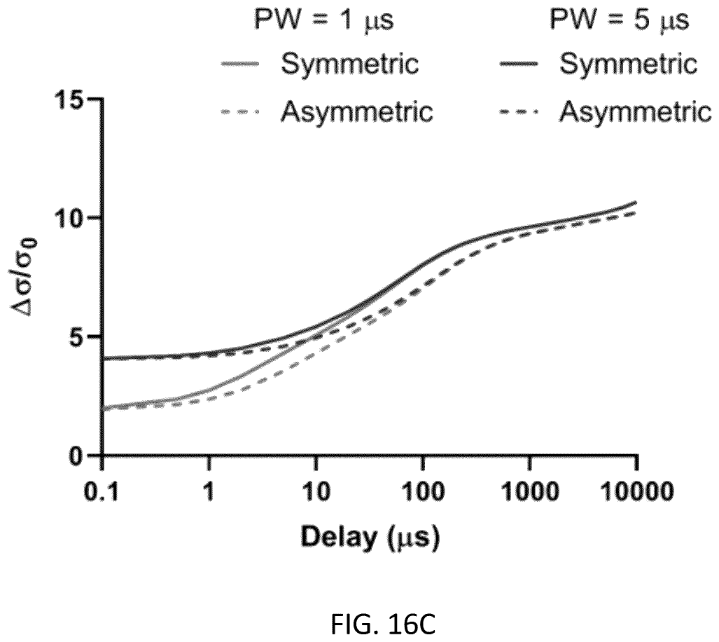

An additional benefit that may be realized by extending d₂ is the reduced effective frequency of the burst (FIGS. 16A-C). The effective frequency is calculated according to:

$${f}_{c} = {( 2 {T}_{p} + {d}_{1} + {d}_{2} )}^{- 1} \qquad ( Equation 7 )$$

Thus, prolonging d₂ has an inverse effect on f_{c}, reductions of which are thought to correlate with reductions in electrical conductivity according to the impedance spectrum of the tissue of interest. Assuming that any given burst waveform with identical amplitude, constitutive pulse width, and energy will result in the same extent of permeabilization with enough applied pulses, it can be inferred that extensions in d₂ result in greater relative changes in conductivity (FIGS. 16A-C) due to electroporation and, thus, greater exposure to therapeutic electric fields. FIGS. 16A-C demonstrate the effect of modifying delays on the electrical characteristics of the pulse, including the characteristic frequency, baseline conductivity, and fold change in conductivity. Notably, the magnitude of change in conductivity correlates with the increased exposure of the tissue to therapeutic fields. Thus, larger relative increases in conductivity assist the redistribution of the field to expose more tissue to therapeutic electric field strengths.

By maximizing the interpulse delay using the disclosed methods, the user has the ability to improve the effects of treatment while lowering the risk of side effects such as tissue excitation and thermal damage. Table V gives representative ranges for each parameter in the burst.

TABLE V
Representative values for various parameters of the waveform.

| | | Intra- | | Inter- | Bipolar | | | | |
|---|---|---|---|---|---|---|---|---|---|
| | | phase | | pulse | Pulse | Energized | Burst | Effective | Repetition |
| | (+)PW | Delay | (−)PW | Delay | Period | Time | Width | Frequency | Rate |
| | (μs) | (μs) | (μs) | (μs) | (μs) | (μs) | (ms) | (kHz) | (min⁻¹) |
| Symmetric | 1 | 0.5 | 1 | 0.5 | 3 | 60 | 0.0895 | 333.333 | 30-90 |
| | 1 | 1 | 1 | 1 | 4 | 60 | 0.119 | 250.000 | 30-90 |
| | 1 | 2 | 1 | 2 | 6 | 60 | 0.178 | 166.667 | 30-90 |
| | 1 | 5 | 1 | 5 | 12 | 60 | 0.355 | 83.333 | 30-90 |
| | 1 | 10 | 1 | 10 | 22 | 60 | 0.65 | 45.455 | 30-90 |
| | 1 | 100 | 1 | 100 | 202 | 60 | 5.96 | 4.950 | 30-90 |
| | 1 | 1000 | 1 | 1000 | 2002 | 60 | 59.06 | 0.500 | 30-90 |
| Asymmetric | 1 | 1 | 1 | 0.25 | 3.25 | 60 | 0.09725 | 307.692 | 30-90 |
| | 1 | 1 | 1 | 1 | 4 | 60 | 0.119 | 250.000 | 30-90 |
| | 1 | 1 | 1 | 2 | 5 | 60 | 0.148 | 200.000 | 30-90 |
| | 1 | 1 | 1 | 5 | 8 | 60 | 0.235 | 125.000 | 30-90 |
| | 1 | 1 | 1 | 10 | 13 | 60 | 0.38 | 76.92 | 30-90 |
| | 1 | 1 | 1 | 100 | 103 | 60 | 2.99 | 9.709 | 30-90 |
| | 1 | 1 | 1 | 1000 | 1003 | 60 | 29.09 | 0.997 | 30-90 |
| Symmetric | 1 | 0.5 | 1 | 0.5 | 3 | 80 | 0.1195 | 333.333 | 30-90 |
| | 1 | 1 | 1 | 1 | 4 | 80 | 0.159 | 250.000 | 30-90 |
| | 1 | 2 | 1 | 2 | 6 | 80 | 0.238 | 166.667 | 30-90 |
| | 1 | 5 | 1 | 5 | 12 | 80 | 0.475 | 83.333 | 30-90 |
| | 1 | 10 | 1 | 10 | 22 | 80 | 0.87 | 45.455 | 30-90 |
| | 1 | 100 | 1 | 100 | 202 | 80 | 7.98 | 4.950 | 30-90 |
| | 1 | 1000 | 1 | 1000 | 2002 | 80 | 79.08 | 0.500 | 30-90 |
| Asymmetric | 1 | 1 | 1 | 0.5 | 3.5 | 80 | 0.1395 | 285.714 | 30-90 |
| | 1 | 1 | 1 | 1 | 4 | 80 | 0.159 | 250.000 | 30-90 |
| | 1 | 1 | 1 | 2 | 5 | 80 | 0.198 | 200.000 | 30-90 |
| | 1 | 1 | 1 | 5 | 8 | 80 | 0.315 | 125.000 | 30-90 |
| | 1 | 1 | 1 | 10 | 13 | 80 | 0.51 | 76.92 | 30-90 |
| | 1 | 1 | 1 | 100 | 103 | 80 | 4.02 | 9.709 | 30-90 |
| | 1 | 1 | 1 | 1000 | 1003 | 80 | 39.12 | 0.997 | 30-90 |
| Symmetric | 1 | 0.5 | 1 | 0.5 | 3 | 100 | 0.1495 | 333.333 | 30-90 |
| | 1 | 1 | 1 | 1 | 4 | 100 | 0.199 | 250.000 | 30-90 |
| | 1 | 2 | 1 | 2 | 6 | 100 | 0.298 | 166.667 | 30-90 |
| | 1 | 5 | 1 | 5 | 12 | 100 | 0.595 | 83.333 | 30-90 |
| | 1 | 10 | 1 | 10 | 22 | 100 | 1.09 | 45.455 | 30-90 |
| | 1 | 100 | 1 | 100 | 202 | 100 | 10 | 4.950 | 30-90 |
| | 1 | 1000 | 1 | 1000 | 2002 | 100 | 99.1 | 0.500 | 30-90 |
| Asymmetric | 1 | 1 | 1 | 0.5 | 3.5 | 100 | 0.1745 | 285.714 | 30-90 |
| | 1 | 1 | 1 | 1 | 4 | 100 | 0.199 | 250.000 | 30-90 |
| | 1 | 1 | 1 | 2 | 5 | 100 | 0.248 | 200.000 | 30-90 |
| | 1 | 1 | 1 | 5 | 8 | 100 | 0.395 | 125.000 | 30-90 |
| | 1 | 1 | 1 | 10 | 13 | 100 | 0.64 | 76.92 | 30-90 |
| | 1 | 1 | 1 | 100 | 103 | 100 | 5.05 | 9.709 | 30-90 |
| | 1 | 1 | 1 | 1000 | 1003 | 100 | 49.15 | 0.997 | 30-90 |

The methods described apply to experimental and therapeutic applications in which bursts of pulsed electric fields are employed. The methods allow the user to modify the burst to achieve the desired response while mitigating side effects such as nerve excitation and potentially thermal damage.

In particular, the methods introduced are the first to describe procedures using asymmetric spacing within bursts of bipolar pulses and/or techniques for maximizing delays using the refractory periods of either cardiac or skeletal myocytes as a guideline.

A modified delay structure has been developed which capitalizes on the benefits of both burst and continuous delivery. These methods describe the use of maximal spacing between subsequent pulses (interpulse delay) such that summative effects on nerve excitation within the burst are reduced. In addition to limiting nerve excitation, these extended delays modulate the characteristic frequency at which the burst operates, which reduces the baseline electrical conductivity and, as a result, the extent of Joule heating. This reduced initial conductivity also increases exposure to therapeutic electric field strengths. Also, by modulating the intraphase delay, may allow one to modify ablation size due to changes in the total time of exposure of targeted cells to a critical transmembrane potential of 1 V.

The methods herein describe modifications to pulsed electric field waveforms that may enhance their biological and clinical outcomes while mitigating side effects such as nerve stimulation. Traditionally, bursts of bipolar pulses were developed with relatively short, symmetric delays between pulses in the waveform simply to mitigate assisted discharge between subsequent pulses of alternating polarity and to provide a protective barrier for MOSFET switching. Translation of this approach to monopolar pulses has also been described with the same rationale. The methods introduced herein allow the user to select the delays within these delivery strategies such that the total energy delivered is equivalent, while the extent of muscle stimulation is reduced and biophysical effects are maintained or magnified.

### Example 5

Treatment of tissue with IRE and H-FIRE based technologies involves the administration of high-voltage electrical pulses across electrode pairs inserted in and/or around the tissue of interest. Pulse delivery results in production of high electric fields, especially near electrodes, which result in the electrolytic breakdown of water into hydrogen gas and hydroxide ions at the cathode, with oxygen gas and hydrogen ions accumulating at the anode. Due to ion accumulation, the anode tends to undergo a change in pH to become quite acidic, while significant gas accumulation has been noted at the cathode (Guenther, E. et al., “Electrical breakdown in tissue electroporation,” Biochem. Biophys. Res. Commun., vol. 467, no. 4, 736-741, November 2015; Rubinsky, L. et al., “Electrolytic Effects During Tissue Ablation by Electroporation,” Technol. Cancer Res. Treat., vol. 15, no. 5, NP95-103, 2016.).

Because oxygen gas dissolves easily in water, gas production is usually not an issue at the anode. However, bubbles accumulating and remaining at the cathode may cause distortions to the electric field distribution. Additionally, if bubbles are sufficiently large, subsequent pulses administered in the presence of the bubbles can cause them to break down, resulting in stray and unpredictable currents, as well as potentially causing equipment damage, treatment termination, and patient safety concerns.

This dielectric breakdown has been visualized experimentally and may be associated with the pulsing noises noted in some procedures (Guenther, 2015). Gas production has been noted in clinical IRE procedures for oncological applications, as well as for H-FIRE and IRE procedures used for cardiac ablation (A. Verma et al., “Primer on Pulsed Electrical Field Ablation: Understanding the Benefits and Limitations,” Circ. Arrhythmia Electrophysiol., no. September, pp. 1-16, 2021; T. J. Buist et al., “Efficacy of multi-electrode linear irreversible electroporation,” Europace, vol. 23, no. 3, pp. 464-468, 2021). Importantly, depending upon the electrode configuration and material, as well as the voltage employed, both bipolar and monopolar pulses and/or bursts are subject to bubble production. When significant gas is produced, it can be easily visualized on intraoperative ultrasound, and in some cases may make it more difficult to visualize electrodes and other structures within the region of interest.

Notably, bubble production can be harmful directly, especially during cardiac-based treatments, due to the fact that larger bubbles can migrate to the cerebral microvasculature, where they pose a risk of becoming embolic and creating regions of ischemia (Verma, 2021). As noted above, if enough bubbles are produced, or if they are sufficiently large, application of subsequent pulses poses the risk of bubble dielectric breakdown. This process, also known as arcing, has gained some attention in the literature but has not been rigorously investigated (Guenther, 2015; Buist, 2021; T. J. O'Brien et al., “Effects of internal electrode cooling on irreversible electroporation using a perfused organ model,” Int. J. Hyperth., vol. 35, no. 1, pp. 44-55, 2019; Wandel, 2016). However, if arcing does occur during treatment, it poses several concerns.

First, arcing is highly uncontrolled and can lead to stray currents within the patient and potentially pose an increased risk of generating cardiac arrhythmias. Also, the high currents associated with dielectric breakdown could damage generator electronics, rendering the treatment incomplete prematurely. Electrical arcing can also lead to tissue damage due to pressure waves generated by bubble collapse (FIG. 2A) (Guenther, 2015). Thus, nerves and/or vasculature near the electrodes could be physically damaged, causing a penumbra of possible side effects. Finally, the efficacy of IRE is highly dependent upon the electric field distribution and adequate exposure of target tissue to a critical number of pulses above a certain field threshold. If significant tissue damage due to bubble creation/collapse is occurring near the electrodes, it is possible these regions are disrupting the field distribution, which could lead to undertreatment or otherwise unexpected ablation geometries. For these reasons, reducing the likelihood of gas bubble production and/or arcing has significant advantages for clinicians and patients alike, and may enhance the consistency of treatments.

The interpulse delay within H-FIRE bursts plays a role in the likelihood of this electrical arcing (also termed dielectric breakdown). Arcing is likely the result of applying high voltage pulses in the presence of a sufficient number of hydrogen gas bubbles on the cathode. When increasing the interpulse delay for example from 5 μs to 250 μs (2-5-2-5 vs 2-5-2-250), electric potentials over 500 V higher than with traditional waveforms were able to be applied prior to the onset of electrical breakdown. It is believed that the interpulse (and by extrapolation, interphase) delay allows for one of several possible effects to occur that might explain the reduced tendency of arcing to occur with long delays. It is expected that this finding will also translate to monopolar pulses, so longer delays built into monopolar bursts should also be advantageous from this perspective.

Although hydrogen gas does not dissolve quickly in water, it is possible that dissolution of a small number of bubbles is occurring with longer delays, which reduces the potential for arcing. With longer interpulse delays, hydrogen bubbles may be diffusing away from the electrodes, where fields (and current densities) are highest. If sufficient diffusion is occurring within the delay period to disperse bubbles such that they are not as concentrated, they may be less likely to coalesce and impede current flow, thus reducing the likelihood of arcing. It is worth noting that hydrogen gas is quite mobile relative to other elemental gases mostly due to its small hydrodynamic radius. Another mechanism possibly accounting for the results is based on bubble production. It is possible that with short delays, each pulse within a burst could generate more electrolytic products (gas), and this could create synergy within the burst, wherein gas bubbles grow, coalesce, and/or expand with each subsequent pulse throughout the burst duration. With longer delays built into the burst, these dynamics might be disrupted, preventing synergistic effects due to bubbles having sufficient time to stabilize after each pulse, and thus, limiting the likelihood for arcing.

Regardless of the mechanisms accounting for the effects observed experimentally, it is anticipated that increasing the interpulse (or interphase) delay will directly decrease the risk of arcing with all other factors (geometric and pulse paradigm) remaining identical. The utility of this finding is that the delays within a waveform can be selected to improve treatment predictability and to maximize patient safety, regardless of waveform type, electrode configuration, and clinical application.

For example, a clinician may choose to extend d₁ and/or d₂ to mitigate the risk of arcing. A clinician may also choose a shorter time period for d₁ or a longer time period for d₂ to reduce/prevent muscle contractions. In embodiments, a clinician may choose a burst interval that falls within the absolute refractory period. Example pulse protocols a clinician may choose to mitigate bubble formation and/or electrical arcing are shown in Tables VI-VIII.

TABLE VI
Pulse protocols to mitigate bubble formation

| | Pulse or | | Pulse or | |
|---|---|---|---|---|
| Example | Phase 1 | d₁ | Phase 2 | d₂ |
| 1 | 1 | 1 | 1 | 1000 |
| 2 | 2 | 10 | 2 | 2000 |
| 3 | 5 | 2 | 5 | 200 |
| 4 | 5 | 1 | 5 | 10 |
| 5 | 5 | 10 | 5 | 100 |
| 6 | 10 | 10 | 10 | 5000 |
| 7 | 10 | 2 | 10 | 1000 |
| 8 | 5 | 10 | 5 | 1000 |
| 9 | 1 | 2 | 1 | 100 |
| 10 | 2 | 5 | 2 | 1000 |

TABLE VII
Example values of waveform parameters within bipolar burst

| Pulse | Interphase | Pulse | Interpulse | Bipolar |
|---|---|---|---|---|
| duration \| | delay | duration \| | delay | pulse |
| polarity (μs) | (d₁, μs) | polarity (μs) | (d₂, μs) | period (μs) |
| 0.5 − 10 \| + | 0.5 − 10,000 | 0.5 − 10 \| − | 0.5 − 10,000 | 2 − 20,020 |
| 0.5 − 10 \| − | 0.5 − 10,000 | 0.5 − 10 \| + | 0.5 − 10,000 | 2 − 20,020 |

TABLE VIII
Example values of waveform parameters within monopolar burst

| Pulse duration \| | Interpulse | Monopolar pulse |
|---|---|---|
| polarity (μs) | delay (μs) | period (μs) |
| 0.5 − 10 \| + | 0.5 − 10,000 | 1 − 10,010 |
| 0.5 − 10 \| − | 0.5 − 10,000 | 1 − 10,010 |

The present invention has been described with reference to particular embodiments having various features. In light of the disclosure provided herein, it will be apparent to those skilled in the art that various modifications and variations can be made in the practice of the present invention without departing from the scope or spirit of the invention. One skilled in the art will recognize that the disclosed features may be used singularly, in any combination, or omitted based on the requirements and specifications of a given application or design. When an embodiment refers to “comprising” certain features, it is to be understood that the embodiments can alternatively “consist of” or “consist essentially of” any one or more of the features. Other embodiments of the invention will be apparent to those skilled in the art from consideration of the specification and practice of the invention.

It is noted in particular that where a range of values is provided in this specification, each value between the upper and lower limits of that range is also specifically disclosed. The upper and lower limits of these smaller ranges may independently be included or excluded in the range as well. The singular forms “a,” “an,” and “the” include plural referents unless the context clearly dictates otherwise. It is intended that the specification and examples be considered as exemplary in nature and that variations that do not depart from the essence of the invention fall within the scope of the invention. Further, all of the references cited in this disclosure are each individually incorporated by reference herein in their entireties and as such are intended to provide an efficient way of supplementing the enabling disclosure of this invention as well as provide background detailing the level of ordinary skill in the art.

1. A method of applying electrical energy to tissue, the method comprising:
administering to a tissue a series of bursts of bipolar electrical pulses, wherein each bipolar electrical pulse comprises a positive phase and a negative phase, wherein each phase has a duration of 250 ns to 250 μs, with or without a delay between the positive and negative phases;
wherein each bipolar electrical pulse is separated by a delay lasting up to 1 ms;
wherein the bipolar electrical pulses are repeated until a total energized time of 1 μs to 500 μs is achieved;
wherein one or more of the bursts has a burst width of at least 1 μs;
wherein the series of bursts is administered for a total time of 250 ms to 10 seconds; and
wherein the bipolar electrical pulses are applied at a voltage ranging from 500 V to 10 kV.

2. The method of claim 1, wherein the series of bursts is administered in a manner to provide reversible electroporation or irreversible electroporation.

3. The method of claim 1, wherein where cardiac synchronization is desired one or more of the bursts has a burst width of 10 μs to 300 ms.

4. The method of claim 1, wherein each phase has a duration of 250 ns to 10 μs and wherein the bipolar electrical pulses are applied at a voltage ranging from 1 kV to 5 kV.

5. The method of claim 1, wherein one or more of the bursts has a burst width of 100 μs.

6. The method of claim 1, wherein there is a delay between one or more of the positive phases and the negative phase.

7. The method of claim 6, wherein the delay between the positive phase and the negative phase is shorter than the delay between each bipolar electrical pulse.

8. A method for applying electrical energy to tissue, the method comprising:
administering to a tissue a series of bursts of positive monopolar electrical pulses;
wherein each of the positive pulses has a duration of 250 ns to 250 μs;
wherein each burst comprises one or more delay of up to 100 ms;
wherein the positive pulses are repeated until a total energized time of 1 μs to 500 μs is achieved;
wherein the burst interval of at least two of the bursts is between 250 ms and 10 seconds;
and wherein the positive pulses are applied at a voltage ranging from 25 V to 10 kV.

9. The method of claim 8, wherein the series of bursts is administered to provide reversible electroporation or irreversible electroporation.

10. The method of claim 8, wherein the positive pulses are applied at a voltage ranging from 500 V to 5 kV.

11. The method of claim 8, wherein the positive pulses each have a duration of 250 ns to 10 μs.

12. The method of claim 8, wherein a burst is administered once every 0.5 s to 3 s.

13. The method of claim 8, wherein at least one of the bursts has a burst width of 100 μs.

14. A method of applying electrical energy to tissue, the method comprising:
applying one or more burst of a plurality of electrical pulses to a tissue;
wherein one or more of the bursts comprises energy with positive phases and/or negative phases;
wherein one or more parameters of the burst are selected such that the burst is capable of causing electroporation of the tissue in a manner that avoids, prevents, minimizes or suppresses neural excitation, bubble formation, and/or Joule heating.

15. The method of claim 14, wherein one or more of the parameters is that a time between one of the pulses and another of the pulses is longer than a time between one of the positive phases and one of the negative phases.

16. The method of claim 14, wherein the time between one of the positive phases and one of the negative phases comprises an interphase delay ranging from 0 to 5 μs.

17. The method of claim 14, wherein the time between one of the electrical pulses and another of the electrical pulses comprises an interpulse delay in the range of 10 μs to 1 second.

18. The method of claim 17, wherein the interpulse delay is 5 to 1,000 times longer than an interphase delay of the electrical pulses.

19. The method of claim 14, wherein one or more of the bursts has a total energized time of from 1 μs to 1 s.

20. The method of claim 14, wherein one or more of the pulses, one or more of the positive phases, and/or one or more of the negative phases have a length of up to 10 μs.
